SEQUENCE LISTING (17 sequences)

SEQ ID NO: 1 - RNA, 499 nt, Candida albicans
cggguaaacggcgggaguaacuaugacucucaaccuauaagggaggcaaaaguagggacgccaugguuuccagaaaugggccgcgguguuuuugaccugcuagucgaucuggccagaaguaucuguggguggccagcggcgacauaaccugguacggggaaggccucgaagcaguguucaccuugggagugcgcaagcacaaagaggugagugguguaugggguuaaucccguggcgagccgucagggcgcgaguucuggcaguggccgucguagagcacggaaagguaugggcuggcucucugagucggcuuaagguacgugccgucccacacgaugaaaagugugcggugcagaauaguucccacagaacgaagcugcgccggagaaagcgauuucuuggagcaaugcuuaaaguagccaaaugccucgucaucuaauuagugacgcgcgcaugaguggauuaacgagauucccacugucccuaucuaucuacuauc

SEQ ID NO: 2 - RNA, 12 nt, Artificial Sequence
  Description of Artificial Sequence oligonucleotide
ucuacgacggcc

SEQ ID NO: 3 - DNA, 12 bp, Artificial Sequence
  Description of Artificial Sequence oligonucleotide
tntncnangncn

SEQ ID NO: 4 - DNA, 12 bp, Artificial Sequence
  Description of Artificial Sequence oligonucleotide
antngnantngn

SEQ ID NO: 5 - DNA, 8 bp, Artificial Sequence
  Description of Artificial Sequence oligonucleotide
nnnnnnnn

SEQ ID NO: 6 - DNA, 8 bp, Artificial Sequence
  Description of Artificial Sequence oligonucleotide
nnnnnnnn

SEQ ID NO: 7 - DNA, 8 bp, Artificial Sequence
  Description of Artificial Sequence oligonucleotide
nnnnnnnn

SEQ ID NO: 8 - RNA, 6 nt, Artificial Sequence
  Description of Artificial Sequence oligonucleotide
gacucu

SEQ ID NO: 9 - RNA, 12 nt, Artificial Sequence
  Description of Artificial Sequence oligonucleotide
uuuuuugacucu

SEQ ID NO: 10 - RNA, 8 nt, Artificial Sequence
  Description of Artificial Sequence oligonucleotide
nannnnnn

SEQ ID NO: 11 - RNA, 377 nt, Escherichia coli
gaagcugaccagacagucgccgcuucgucgucguccucuucgggggagacgggcggaggggaggaaaguccgggcuccauagggcagggugccagguaacgccugggggggaaacccacgaccagugcaacagagagcaaaccgccgauggcccgcgcaagcgggaucagguaagggugaaagggugcgguaagagcgcaccgcgcggcugguaacaguccguggcacgguaaacuccacccggagcaaggccaaauagggguucauaagguacggcccguacugaacccggguaggcugcuugagccagugagcgauugcuggccuagaugaaugacuguccacgacagaacccggcuuaucggucaguuucaccu

SEQ ID NO: 12 - RNA, 12 nt, Artificial Sequence
  Description of Artificial Sequence oligonucleotide
cagccuacccgg

SEQ ID NO: 13 - DNA, 8 bp, Artificial Sequence
  Description of Artificial Sequence oligonucleotide
tacctttc

SEQ ID NO: 14 - DNA, 12 bp, Artificial Sequence
  Description of Artificial Sequence oligonucleotide
tctacgacggcc

SEQ ID NO: 15 - RNA, 8 nt, Artificial Sequence
  Description of Artificial Sequence oligonucleotide
uaccuuuc

SEQ ID NO: 16 - RNA, 12 nt, Artificial Sequence
  Description of Artificial Sequence oligonucleotide
agaugcugccgg

SEQ ID NO: 17 - RNA, 8 nt, Artificial Sequence
  Description of Artificial Sequence oligonucleotide
auggaaag

(19) United States
(12) Patent Application Publication
(10) Pub. No.: US 2004/0030111 A1
(43) Pub. Date: Feb. 12, 2004

(54) Oligonucleotide directed misfolding of RNA

(76) Inventors: Douglas H. Turner, Pittsford, NY (US); Jessica L. Childs, Zurich (CH); Matthew D. Disney, Zurich (CH)

Correspondence Address: Edwin V. Merkel, NIXON PEABODY LLP, Clinton Square, P.O. Box 31051, Rochester, NY, 14603-1051, US

(21) Appl. No.: 10/465,730

(22) Filed: Jun. 19, 2003

Related U.S. Application Data
(60) Provisional application No. 60/390,241, filed on Jun. 19, 2002.

(51) Int. Cl. C07H 21/02; A61K 48/00

(52) U.S. Cl. 536/23.1; 514/44

(57) ABSTRACT

Oligonucleotides that bind to and cause misfolding of functional RNA molecules are described. Also disclosed are the uses of the oligonucleotides to modify the function of such RNA molecules, to stabilize the RNA molecules in a misfolded conformation, to disrupt survivability of a pathogen or cancer cells (that require activity of the RNA molecule for survival) by disrupting the activity of the RNA molecules, treating or preventing pathogen infection in a patient, and treating a cancerous condition in a patient. Methods of designing the oligonucleotides of the present invention are also disclosed.

This application claims the priority benefit of U.S. Provisional Patent Application Serial No. 60/390,241 filed Jun. 19, 2002, which is hereby incorporated by reference in its entirety.

This invention was made, at least in part, with funding received from the National Institutes of Health grant numbers GM22939 and T32 DE07202. The U.S. government may have certain rights in this invention.

### FIELD OF THE INVENTION

This invention relates generally to oligonucleotides and their use in directing the misfolding of functional RNA molecules that are characterized by secondary and/or tertiary folding to achieve an active conformation, whereby the misfolding modifies the activity of the RNA molecules.

### BACKGROUND OF THE INVENTION

RNA is emerging as an important target for therapeutics (Hermann et al., Curr. Opin. Biotechnol. 9:66-73 (1998); Pearson et al., Chem. Biol. 4:409-414 (1997)). For example, antisense oligonucleotides, including Vitravene™ (Galderisi et al., J. Cell. Physiol. 181:251-257 (1999)) and Gentasense™ (Thayer, Chem. Eng. News 80:10 (2002); Stein, Nat. Biotechnol. 19:737-738 (2001)), are proving effective (Agrawal, Trends Biotechnol. 14:376-387 (1996); Holmlund et al., Curr. Opin. Mol. Ther. 1:372-385 (1999)). Oligonucleotides are a promising class of therapeutics because they can be designed from simple base pairing rules and they have pharmacokinetic properties that are relatively independent of sequence (Crooke et al., J. Pharmacol. Exp. Ther. 277:923-937 (1996)). Furthermore, many analogs are synthetically accessible (Freier et al., Nucleic Acids Res. 25:4429-4443 (1997)). Currently, oligonucleotides in the clinic rely on formation of about 20 base pairs between oligonucleotide and target RNA. Recent insights into RNA folding, however, suggest that RNA can be targeted specifically with shorter oligonucleotides (Bevilacqua et al., Biochemistry 30:10632-10640 (1991); Testa et al., Proc. Natl. Acad. Sci. USA 96:2734-2739 (1999); and Disney et al., Biochemistry 40:6507-6519 (2001)).

Many RNAs require proper tertiary folds to function. Examples include rRNAs (Noller et al., Science 256:1416-1419 (1992); Nissen et al., Science 289:920-930 (2000); Carter et al., Science 291:498-501 (2001)), mRNAs with 5′ UTRs that regulate translation (Miranda-Rios et al., Proc. Natl. Acad. Sci. USA 98:9736-9741 (2001); Kim et al., Proc. Natl. Acad. Sci. USA 96:14234-14239 (1999); and Tang et al., Cell 57:531-536 (1989)), RNase P (Houser-Scott et al., Methods Enzymol. 342:101-117 (2001); Waugh et al., Science 244:1569-1571 (1989); Stark et al., Proc. Natl. Acad. Sci. USA 75:3717-3721 (1978)), and group I (Cech et al., in The RNA World, 2nd ed., Gesteland et al., Eds, pp. 321-349, Cold Spring Harbor Laboratory Press, Cold Spring Harbor, N.Y. (1999); Michel et al., J. Mol. Biol. 216:585-610 (1990)) and group II introns (Peebles et al., Cell 44:213-223 (1986); Su et al., J. Mol. Biol. 306:655-668 (2001); Ferat et al., Nature 364:358-361 (1993)). These RNAs can have both active and inactive folds that are similar in free energy, and the inactive folds can be trapped kinetically (Walstrum et al., Biochemistry 29:10573-10576 (1990); Uhlenbeck, RNA 1:4-6 (1995); Pan et al., J. Mol. Biol. 280:597-609 (1998); Treiber et al., Science 279:1943-1946 (1998); Chadalavada et al., J. Mol. Biol. 301:349-367 (2000)). Kinetic traps that cause inactivation of catalytic RNAs have been observed, for example, in studies of the Tetrahymena thermophila group I intron (Walstrum et al., Biochemistry 29:10573-10576 (1990); Pan et al., J. Mol. Biol. 280:597-609 (1998); Celander et al., Science 251:401-407 (1991); Russell et al., Proc. Natl. Acad. Sci. USA 99:155-160 (2002)), the hammerhead ribozyme (Fedor et al., Proc. Natl. Acad. Sci. USA 87:1668-1672 (1990)), and the hepatitis delta virus ribozyme (Chadalavada et al., J. Mol. BioL 301:349-367 (2000); Been et al., Biochemistry 31:11843-11852 (1992)). These traps are often the result of secondary structure rearrangement. Secondary structure prediction (Mathews et al., J. Mol. Biol. 288:911-940 (1999)) can give insights into possible inactive folds that can lead to kinetic traps (Pan et al., J. Mol. Biol. 280:597609 (1998); Chadalavada et al., J. Mol. Biol. 301:349-367 (2000); Banerjee et al., Biochemistry 34:6504-6512 (1995)). Though kinetic traps may be disadvantageous for folding studies, it would be desirable to identify an approach that can utilize kinetic traps to modify, typically inhibit in whole or in part, the activity of functional RNA molecules.

The present invention is directed to overcoming these and other deficiencies in the art.

### SUMMARY OF THE INVENTION

A first aspect of the present invention relates to a method of modifying the function of an RNA molecule that includes: providing an oligonucleotide and binding the oligonucleotide to an RNA molecule that possesses an activity at least partially dependent on secondary and/or tertiary folding thereof into an active conformation, wherein said binding occurs at a site of the RNA molecule that prevents folding thereof into the active conformation and thereby modifies the function of the RNA molecule.

A second aspect of the present invention relates to a method of stabilizing an RNA molecule in a substantially inactive conformation. This method of the present invention includes: binding an oligonucleotide to an RNA molecule at a site of the RNA molecule that causes the RNA molecule to adopt a substantially inactive conformation that is distinct of an active conformation thereof, wherein the RNA molecule is initially stabilized in the substantially inactive conformation by said binding.

A third aspect of the present invention relates to an isolated oligonucleotide that includes a nucleotide sequence that binds to at least one domain of an RNA molecule that requires folding to achieve an active conformation that includes a secondary and/or tertiary structure, wherein binding of the oligonucleotide to the RNA molecule inhibits formation of the secondary and/or tertiary structure. The oligonucleotide can be either an oligoRNA or an oligoDNA. Also disclosed are DNA constructs and expression vectors that encode an oligoRNA of the present invention.

A fourth aspect of the present invention relates to a method of disrupting survivability of a pathogen. This method of the present invention includes: providing an oligonucleotide that binds to an RNA molecule of the pathogen, which RNA molecule is characterized by secondary and/or tertiary folding to achieve an active conformation required for pathogen survivability; and binding the oligonucleotide to the RNA molecule, whereby said binding causes the RNA molecule to misfold, thereby disrupting the activity of the RNA molecule and survivability of the pathogen.

A fifth aspect of the present invention relates to a method of treating or preventing pathogen infection in a patient. This method of the present invention includes: providing an oligonucleotide that binds to an RNA molecule of a pathogen, which RNA molecule is characterized by secondary and/or tertiary folding to achieve an active conformation required for pathogen survivability; and administering the oligonucleotide to a patient under conditions effective to cause uptake of the oligonucleotide by the pathogen or patient cells infected with the pathogen, whereby the oligonucleotide binds to the RNA molecule and said binding causes the RNA molecule to misfold, thereby disrupting survivability of the pathogen to treat or prevent pathogen infection in the patient.

A sixth aspect of the present invention relates to a method of disrupting survival or proliferation of a cancer cell. This method of the invention includes: providing an oligonucleotide that binds to an mRNA molecule overexpressed in a cancer cell, wherein overexpression of the mRNA is required for survival or proliferation of the cancer cell; and binding the oligonucleotide to the mRNA molecule, whereby said binding causes the mRNA molecule to misfold, thereby disrupting activity of the mRNA molecule and either survival or proliferation of the cancer cell.

A seventh aspect of the present invention relates to a method of treating a cancerous condition in a patient. This method of the present invention includes: providing an oligonucleotide that binds to an mRNA molecule overexpressed in a cancer cell, wherein overexpression of the mRNA is required for survival or proliferation of the cancer cell; and administering the oligonucleotide to a patient under conditions effective to cause uptake of the oligonucleotide by the cancer cell, whereby the oligonucleotide binds to the mRNA molecule and said binding causes the mRNA molecule to adopt a misfolded conformation, thereby disrupting activity of the mRNA molecule and either survival or proliferation of the cancer cell, which treats the cancerous condition.

An eighth aspect of the present invention relates to a method of making an oligonucleotide that directs misfolding of an RNA molecule into a conformation having modified activity. This method of the present invention includes: predicting the folding structure of an RNA molecule that is characterized by secondary and/or tertiary folding to achieve an active conformation; designing an oligonucleotide to hybridize to the RNA molecule at a site critical for the secondary and/or tertiary folding; and determining whether binding of the oligonucleotide at the site modifies folding of RNA molecule and thereby modifies the activity of the RNA molecule.

The present invention offers a method of controlling the activity of functional RNA molecules characterized by secondary and/or tertiary folding to achieve active conformations. By identifying oligonucleotides that can hybridize to the functional RNA molecules and stabilize those RNA molecules in misfolded conformations (i.e., relative to the active conformation), the oligonucleotides can be used to modify—typically inhibit—activity of the functional RNA molecules and thereby provide phenotypic changes. This is achieved by targeting functional RNAs preferably at the earliest time—during or soon after transcription (i.e., before the RNA has folded into its active conformation)—using small oligonucleotides to direct misfolding of the RNA. The present invention therefore offers preventative or therapeutic oligonucleotide treatments that can be used to control the activity of pathogen RNAs in hosts or other environments (and thereby reduce the survivability of the pathogen). In addition, cancerous conditions can be treated using oligonucleotide directed misfolding to interfere with activity of mRNAs that are overexpressed in cells and required for cancer cell survival or proliferation. The present application provides demonstrated results that show the oligonucleotide directed misfolding of Candida albicans group I intron and E. coli RNase P RNA into non-functional folds. This Oligonucleotide Directed Misfolding of RNA (“ODMiR”) method should be broadly applicable to functional RNAs whose activity can depend at least in part upon secondary and/or tertiary folding.

### BRIEF DESCRIPTION OF THE DRAWINGS

FIGS. 1A-B illustrate the secondary structure of the C. albicans group I intron (SEQ ID No: 1) in its active secondary structure (1A) (Disney et al., Biochemistry 40:6507-6519 (2001), which is hereby incorporated by reference in its entirety) and in a misfolded secondary structure (1 B) as predicted by RNAStructure. In FIG. 1B, brackets denote the misfolded region. The intron and truncated exons are depicted in uppercase and lowercase letters, respectively. Arrows point to the splice sites. The C-10/1× ribozyme starts at G11 and ends at U377 as indicated by boxed letters. The 5′ and 3′ exon deoxyribonucleotides in bold are endogenous to the vector (and are excluded from SEQ ID NO: 1).

FIG. 2 illustrates chemical structures of nucleotides used in this study.

FIGS. 3A-D illustrate the inhibition of C. albicans group I intron self-splicing via oligonucleotide directed misfolding of RNA during transcription. FIGS. 3A-B are autoradiograms of gels for reactions in the presence or absence of m(UCUACGACGGCC) (SEQ ID NO: 2) and T^{L}CT^{L}AC^{L}GA^{L}CG^{L}GC^{L}C (SEQ ID NO: 3), respectively. FIGS. 3C-D are plots of the percentage of intron (◯) and precursor (●) as a function of m(UCUACGACGGCC) (SEQ ID NO: 2) and T^{L}CT^{L}AC^{L}GA^{L}CG^{L}GC^{L}C (SEQ ID NO: 3) concentration, respectively. The control oligonucleotide, A^{L}CT^{L}CG^{L}CA^{L}GT^{L}CG^{L}C (SEQ ID NO: 4), inhibits splicing only at concentrations >10 μM. All concentrations have error bars, though in some instances they are smaller than the data point.

FIGS. 4A-B illustrate the inhibition of C. albicans group I intron self-splicing via oligonucleotide directed misfolding of RNA during transcription. FIG. 4A is an autoradiogram of a gel for transcriptions in the presence or absence of ^{L}(TACCTTTC) (SEQ ID NO: 5). FIG. 4B is a plot of the percentage of intron (◯) and precursor (●) as a function of ^{L}(TACCTTTC) (SEQ ID NO: 5). The control sequences, ^{L}(CCTTATCT) (SEQ ID NO: 6) and ^{L}(ACTCACCT) (SEQ ID NO: 7), inhibit splicing only at concentrations >10 μM. All points have error bars, though in some instances they are smaller than the data points.

FIG. 5 illustrates the misfolding of ribozyme detected by native gel electrophoresis. Lanes: A, ribozyme only; B, r(GACUCU) (SEQ ID NO: 8); C, r(U₆GACUCU) (SEQ ID NO: 9); D, m(UCUACGACGGCC) (SEQ ID NO: 2); E, T^{L}CT^{L}AC^{L}GA^{L}CG^{L}GC^{L}C (SEQ ID NO: 3); F, A^{L}CT^{L}CG^{L}CA^{L}GT^{L}CG^{L}C (SEQ ID NO: 4); G, d(^{P}UA^{P}C^{P}C^{P}U^{P}U^{P}U^{P}C) (SEQ ID NO: 10); H, ^{L}(TACCTTTC) (SEQ ID NO: 5); I, ^{L}(CCTTATCT) (SEQ ID NO: 6); J, ^{L}(ACTCACCT) (SEQ ID NO: 7). Lanes A, B, and C are standards for properly folded ribozyme. Lane G has an analog of an ODMiR oligonucleotide that has C5-1-propynyl substitutions on all pyrimidines. The d(^{P}UA^{P}C^{P}C^{P}U^{P}U^{P}U^{P}C) (SEQ ID NO: 10) has an IC50 of ˜4 μM. Lanes F and I contain molecules that are not complementary to ribozyme, and thus should not induce a misfold. Lane J has an oligonucleotide complementary to nucleotides 175-183 in P5b (see FIG. 1A).

FIG. 6 illustrates the misfolding of ribozyme detected by DEPC modification. Lanes (a) denote DEPC applied; and (b) denote no DEPC. Lanes: 1, ribozyme annealed with r(GACUCU) (SEQ ID NO: 8) only; 2, with ODMiR oligomer T^{L}CT^{L}AC^{L}GA^{L}CG^{L}GC^{L}C (SEQ ID NO: 3)+r(GACUCU) (SEQ ID NO: 8); 3, with control oligomer A^{L}CT^{L}CG^{L}CA^{L}GT^{L}CG^{L}C (SEQ ID NO: 4)+r(GACUCU) (SEQ ID NO: 8); 4, with ODMiR oligomer ^{L}(TACCTTTC) (SEQ ID NO: 5)+r(GACUCU) (SEQ ID NO: 8); 5, with control oligomer ^{L}(CCTTATCT) (SEQ ID NO: 6)+r(GACUCU) (SEQ ID NO: 8); 6, with control oligomer ^{L}(ACTCACCT) (SEQ ID NO: 7)+r(GACUCU) (SEQ ID NO: 8).

FIG. 7 illustrates the misfolding of ribozyme detected by DEPC modification. Lanes (a) denote DEPC applied; and (b) denote no DEPC. Lanes: 1, ribozyme annealed with r(GACUCU) (SEQ ID NO: 8) only; 2, with ODMiR oligomer T^{L}CT^{L}AC^{L}GA^{L}CG^{L}GC^{L}C (SEQ ID NO: 3)+r(GACUCU) (SEQ ID NO: 8); 3, with control oligomer A^{L}CT^{L}CG^{L}CA^{L}GT^{L}CG^{L}C (SEQ ID NO: 4)+r(GACUCU) (SEQ ID NO: 8).

FIG. 8 illustrates the secondary structure of E. coli RNase P (SEQ ID NO: 11). Tertiary interactions are represented as large boxes connected by dashed lines. The m(CAGCCUACCCGG) (SEQ ID NO: 12) oligonucleotide binds to part of J15/16 as indicated by the boxed nucleotides of this region. Circles represent nucleotides that are more modified by DEPC in the presence of m(CAGCCUACCCGG) (SEQ ID NO: 12).

FIGS. 9A-B illustrate the inhibition of processing of Bacillus subtilis pre-tRNA^{Asp} via oligonucleotide directed misfolding of E. coli RNase P RNA during transcription thereof. FIG. 9A is an autoradiogram of a gel for transcriptions in the presence or absence of m(CAGCCUACCCGG) (SEQ ID NO: 12). FIG. 9B is a plot of the percentage of pre-tRNA (◯) and processed tRNA (tRNA+5′ Leader) (●) as a function of m(CAGCCUACCCGG) (SEQ ID NO: 12) concentration. All points have error bars, though in some instances they are smaller than the data points.

FIG. 10 illustrates the misfolding of RNase P RNA as detected by DEPC modification.

### DETAILED DESCRIPTION OF THE INVENTION

The present invention relates to the binding of oligonucleotides to functional RNA molecules, which possess activities at least partially dependent on secondary and/or tertiary folding thereof into an active conformation, in a manner whereby binding occurs at a site of the RNA molecules that prevents folding thereof into the active conformation and instead causes the RNA molecules to misfold or otherwise adopt a misfolded conformation. By “misfolded”, it is contemplated that the RNA molecules possess a structure having a conformation that is different than the active conformation. As a result of the misfolding, the function of the RNA molecules is modified while they remain misfolded.

An RNA molecule may retain its misfolded conformation while the oligonucleotide remains bound thereto or even after dissociation of the oligonucleotide therefrom. In the former instance, binding of the oligonucleotide is required to induce misfolding and may be required for maintenance of the misfolded conformation. In the latter instance, by contrast, binding of the oligonucleotide is required for misfolding but is not required for maintenance of the misfolded conformation.

As a result of the oligonucleotides binding to the RNA molecules, the misfolded conformation of the RNA molecules can be characterized by either enhanced activity or reduced activity. Typically, the misfolded conformation is characterized by reduced activity. Thus, for catalytic RNAs, the ability to catalyze a reaction (or splicing of itself) is either enhanced or reduced, but typically reduced. For mRNAs that yield a protein product, the level of protein expression (i.e., translation) is either enhanced or reduced, but typically reduced. In addition, where the misfolded conformation of the mRNA affects its proper localization, protein expression levels can also be modified. For rRNAs, their ability to form ribosomes or their involvement in protein synthesis can be modified.

In certain embodiments of the present invention, the misfolded conformation is characterized by a reduced activity level. The conformation characterized by reduced activity preferably possesses as an activity level that is diminished by at least about 10%, more preferably at least about 20% or about 25%. In other embodiments, the conformation characterized by reduced activity is a substantially inactive conformation. By “substantially inactive”, it is intended that the RNA molecule possess a greater than 50% reduction in its activity, preferably greater than 75% reduction in its activity, or more preferably greater than 90% reduction in its activity. In some embodiments, the RNA molecule possesses a greater than 95% reduction in its activity or nearly complete absence of such activity.

In referring to the activity of the RNA molecules, it should be understood that the activity detected is typically (though not exclusively) that of a population of RNA molecules rather a single RNA molecule per se. The population of RNA molecules can contain either substantially all the same RNA molecules (i.e., RNA molecules having the same primary nucleotide sequence but perhaps slightly varied secondary or tertiary folding structures) or a population of different RNA molecules that possess a different primary structure yet a similar oligonucleotide target region (i.e., RNA molecules that are different in primary structure but perhaps have the same activities). Thus, the activity of the total population is modified. In so modifying the activity of the population of RNA molecules, it should also be understood that the activity of any single RNA molecule, while it remains misfolded, can be either completely or partially inhibited, or completely or partially enhanced.

To facilitate the misfolding of the targeted RNA molecule, the binding of the oligonucleotide to the RNA molecule preferably occurs either during or shortly after transcription of the RNA molecule, but preferably before the RNA molecule has had an opportunity to fold into its active conformation. Thus, binding preferably occurs at any time prior to completion of secondary and/or tertiary folding of the RNA molecule into the active conformation, but preferably before folding of the region that contains the oligonucleotide binding site.

As described more fully hereinafter, the binding of the oligonucleotide to the targeted RNA molecule can either occur in vitro (where the present invention is useful for screening new therapeutics) or in vivo (where the oligonucleotide itself is a therapeutic or preventative agent).

In accordance with the present invention, the oligonucleotide used to bind to the targeted RNA molecule is preferably less than about 100 nucleotides in length, more preferably less than about 50, about 40, or about 30 nucleotides in length. Most preferred oligonucleotides of the present invention are less than about 20 nucleotides in length, preferably about 6 to about 15 nucleotides in length.

The oligonucleotides of the present invention can be either RNA or DNA. The oligonucleotides of the present invention can also possess one or more modified bases, one or more modified sugars, one or more modified backbones, or combinations thereof that enhance the affinity of the oligonucleotide to binding site(s) on the RNA molecule. Exemplary forms of modified bases are known in the art and include, without limitation, alkylated bases, alkynylated bases, thiouridine, and Gclamp (Flanagan et al., Proc. Natl. Acad. Sci. USA 30:3513-3518 (1999), which is hereby incorporated by reference in its entirety). Exemplary forms of modified sugars are known in the art and include, without limitation, LNA, 2′-O-methyl, 2′-O-methoxyethyl, and 2′-fluoro (see, e.g., Freier and Attmann, Nucl. Acids Res. 25:44294443 (1997), which is hereby incorporated by reference in its entirety). Exemplary forms of modified backbones are known in the art and include, without limitation, phosphoramidates, thiophosphoramidates, and alkylphosphonates. As is known in the art, the use of modified bases, modified sugars, and modified backbones, either individually or in combination, can enhance the affinity of the oligonucleotide to the binding sites(s) of the RNA molecule. As a result, it is possible to enhance the stability of the misfolded conformation of the RNA molecule to which the oligonucleotide remains bound.

The oligonucleotides of the present invention include any oligonucleotide that can bind to the RNA molecule and thereby modify its folding structure (e.g., secondary and/or tertiary structure) to modify the activity thereof. Preferred oligonucleotides include those that contain a nucleotide sequence that binds to at least one domain of an RNA molecule, which requires secondary and/or tertiary folding to achieve an active conformation that includes a secondary structure (e.g., multibranch loops, interior loops, bulge loops, hairpins, and hairpin loops) and/or a tertiary structure (i.e., formed by interactions between two helices, two unpaired regions, or a helix and an unpaired region), wherein binding of the oligonucleotide to the RNA molecule inhibits formation of the secondary and/or tertiary structure. Exemplary tertiary structures include, without limitation, triple helices and pseudoknots.

The RNA molecule whose function is to be modified can be any RNA molecule that is characterized by one or more secondary and/or tertiary structures and whose activity or function is at least partially dependent on secondary and/or tertiary folding thereof into a “correct” or “active” conformation (that possesses the secondary and/or tertiary structures). Examples of such RNA molecules whose activities can be modified in accordance with the present invention include, without limitation: rRNAs; mRNAs generally and, more specifically, mRNAs that possess an untranslated region (e.g., 5′ or 3′ untranslated region) that regulates translation, localization, or RNA stability as well as mRNAs possessing internal ribosome entry sites (IRES); and ribozymes such as hammerhead ribozymes, hairpin ribozymes, RNase P RNA, and self-splicing introns (e.g., Group I and Group II introns).

rRNAs are involved in ribosome subunit formation and, hence, are critical to protein production. By misfolding rRNA, for example, it is possible to preclude or interfere with either ribosomal subunit formation or, if formed, large subunit association with tRNA or small subunit association with mRNA.

mRNAs that possess a 5′ untranslated region that regulates translation thereof have been identified in eukaryotes, prokaryotes, and viruses. Exemplary mRNAs that possess a 5′ untranslated region that regulates translation of the encoded protein product include, without limitation, human neuronal nitric-acid synthase mRNA (Newton et al., J. Biol. Chem. 278:636-644 (2003), which is hereby incorporated by reference in its entirety), heparin sulfate mRNA (Grobe & Esko, J. Biol. Chem. 277:30699-30706 (2003), which is hereby incorporated by reference in its entirety), mRNA of bacterial thiamin biosynthetic genes (Miranda-Rios et al., Proc. Natl. Acad. Sci. USA 98:9736-9741 (2001), which is hereby incorporated by reference in its entirety), beet western yellow virus mRNA (Kim et al., Proc. Natl. Acad. Sci. USA 96:14234-14239 (1999), which is hereby incorporated by reference in its entirety), HIV-1 virus mRNA (Paillart et al., J. Biol. Chem. 277(8):5995-6004 (2002), which is hereby incorporated by reference in its entirety), and human hsp70 mRNA (Vivinus et al., Eur. J. Biochem. 268:1908-1917 (2001), which is hereby incorporated by reference in its entirety). By targeting oligonucleotides to the 5′ untranslated region of such mRNAs, it becomes possible to inhibit the translation of the encoded protein product. Where the protein product is required for survival of a pathogen, inhibition of protein translation can have deleterious effects and be used for therapeutic or preventative intervention.

mRNAs that posses a 3′ untranslated region that regulates translation or stability thereof have been identified in a number of organisms. Exemplary mRNAs that possess such 3′ untranslated regions include, without limitation, α-globin mRNA, which is important for red blood cell development and function (Waggner and Liebhaber, Exp. Biol. Med. 228:387-395 (2003), which is hereby incorporated by reference in its entirety); vasopressin mRNA, which must be localized to nerve cells (Mohr et al., Eur. J. Neurosci. 13:1107-1112 (2001), which is hereby incorporated by reference in its entirety); Ran mRNA, which is important in lipopolysaccharide signaling (Wong et al., J. Biol. Chem. 276:33129-33138 (2001) which is hereby incorporated by reference in its entirety); β-actin mRNA (Zhang et al., Neuron 31:261-275 (2001), which is hereby incorporated by reference in its entirety); tau mRNA, which is implicated in Alzheimer's Disease (Aronov et al., J. Neurosci. 21:6577-6587 (2001), which is hereby incorporated by reference in its entirety); and utrophin, which must be localized to skeletal muscle cells (Gramolini et al., J. Cell. Biol. 154:1173-1183 (2001), which is hereby incorporated by reference in its entirety). Typically, such 3′ untranslated regions are important for mRNA stability. By altering its structure using oligonucleotide directed misfolding of the mRNA, it is possible to destabilize the mRNA and thereby affect protein expression. Where the protein product is required for survival of a pathogen, inhibition of protein expression can have deleterious effects and be used for therapeutic or preventative intervention.

Additional mRNAs that possess untranslated regions that regulate translation, localization, or RNA stability are known in the art (see, e.g., Pesole et al., Nucleic Acids Res. 30:335-340 (2002), which is hereby incorporated by reference in its entirety) and can be modified in accordance with the present invention.

There are several known classes of ribozymes that are involved in the cleavage and/or ligation of RNA chains. A ribozyme is defined as an enzyme which is made of RNA, most of which work on RNA substrates. The known classes include hairpin ribozymes, hammerhead ribozymes, RNAase P (described below), self-splicing introns (described below), minizymes, and other catalytic RNA molecules.

Group I self-splicing introns (Cech et al., in The RNA World, 2nd ed. (Gesteland et al., Eds.), pp. 321-349, Cold Spring Harbor Laboratory Press, Cold Spring Harbor, N.Y. (1999); Michel et al., J. Mol. Biol. 216:585-610 (1990), each of which is hereby incorporated by reference in its entirety) are present in a number of pathogenic organisms. Importantly, these Group I self-splicing introns have not been found in humans or other mammals. Exemplary pathogenic organisms possessing Group I self-splicing introns include, without limitation, Candida (e.g., C. dubliniensis, C. albicans (Mercure et al., Nucleic Acids Res. 21:1490 (1993), which is hereby incorporated by reference in its entirety)), Pneumocystis (e.g., Pneumocystis carinii (Sogin et al., Nucleic Acids Res. 17:5349-5359 (1989), which is hereby incorporated by reference in its entirety)), Aspergillus (e.g., Aspergillus nidulans (Netzker et al., Nucleic Acids Res. 10:4783-4794 (1982), which is hereby incorporated by reference in its entirety)), Bacillus (e.g., Bacillus anthracis), Staphylococcus (e.g., Staphylococcus aureus), and Naegleria.

The group I intron from C. albicans is located in the large subunit ribosomal RNA (LSU rRNA) precursor, and has been previously characterized (Disney et al., Biochemistry 40:6507-6519 (2001); Mercure et al., Nucleic Acids Res. 21:6020-6027 (1993), each of which is hereby incorporated by reference in its entirety). Self-splicing of group I introns from rRNA genes is essential for maturation of ribosomes (Nikolcheva et al., RNA 3:1016-1027 (1997), which is hereby incorporated by reference in its entirety). Thus, inhibition of self-splicing provides a possible therapeutic approach (Testa et al., Proc. Natl. Acad. Sci. USA 96:2734-2739 (1999); Disney et al., Biochemistry 40:6507-6519 (2001); Mei et al., Bioorg. Med. Chem. 5:1185-1195 (1997); Miletti et al., Antimicrob. Agents Chemother. 44:958-966 (2000), each of which is hereby incorporated by reference in its entirety). By altering Group I intron structure using oligonucleotide directed misfolding of RNA, it is possible to inhibit intron splicing and preclude formation of an RNA that is involved in ribosome maturation. Where the RNA or its protein product is required for survival of a pathogen, inhibition thereof can have deleterious effects and be used for therapeutic or preventative intervention.

Group II self-splicing introns were initially discovered and studied in organelles of plants, fungi and other lower eukaryotes where they are relatively abundant. The introns were found to be catalytic RNAs that self-splice in vitro with a mechanism analogous to nuclear pre-mRNA splicing (see Dai & Zimmerly, Nucleic Acids Res 30:1091-1102 (2002), which is hereby incorporated by reference in its entirety). Some group II introns were observed to encode reverse transcriptase open reading frames (RT ORFs), and were shown to be active retroelements that utilize a mobility mechanism similar to nuclear non-long terminal repeat (non-LTR) retroelements. Group II self-splicing introns have been identified in over 20 bacteria to date, including a number of pathogenic organisms. Importantly, these Group II self-splicing introns have not been found in humans. Exemplary pathogenic organisms possessing Group II self-splicing introns include, without limitation, Escherichia coli, Bacillus anthracis, and Streptococcus pneumoniae (Dai & Zimmerly, Nucleic Acids Res. 30:1091-1102 (2002), which is hereby incorporated by reference in its entirety). To the extent that Group II introns, like Group I introns, lie within an open reading frame of a gene essential to organism survival, inhibition of such Group II intron self-splicing by oligonucleotide directed misfolding of RNA should likewise provide a therapeutic or preventative intervention.

RNase P RNAs are responsible for the site-specific removal of the 5′ leader stem of all pre-tRNAs (Kole et al., Proc. Natl. Acad. Sci. USA 76:3795-3799 (1979); Stark et al., Proc. Natl. Acad. Sci. USA 75:3717-3721 (1978); Altman et al., FASEB J. 7:7-14 (1993); Pace et al., J. Bacteriol. 177:1919-1928 (1995), each of which is hereby incorporated by reference in its entirety) and are essential for cell survival. In E. coli, RNase P RNA is in a 1: f complex with RNase P protein (Talbot et al., Biochemistry 33:1399-1405 (1994); Talbot et al., Biochemistry 33:1406-1411 (1994), each of which is hereby incorporated by reference in its entirety), which is required for in vivo pre-tRNA processing (Altman et al., FASEB J 7:7-14 (1993); Pace et al., J. Bacteriol. 177:1919-1928 (1995), each of which is hereby incorporated by reference in its entirety). In vitro, however, RNase P RNA can catalyze this reaction in the absence of protein when salt concentrations are ˜800 mM NH₄⁺. There is at least one slow step in the folding of E. coli RNase P RNA in which P7 undergoes a conformational change (Zarrinkar et al., RNA 2:564-573 (1996), which is hereby incorporated by reference in its entirety). More details of the folding pathway for Bacillus subtilis RNase P RNA are known, but they are likely to be similar to E. coli RNase P RNA (Zarrinkar et al., RNA 2:564-573 (1996), which is hereby incorporated by reference in its entirety). They reveal that the major kinetic trap involves tertiary interactions between the catalytic (C) and specificity (S) domains (Pan et al., Nat. Struct. Biol. 4:931-938 (1997); Pan et al., J. Mol. Biol. 280:597-609 (1998), each of which is hereby incorporated by reference in its entirety). This trap is eliminated when the domains are folded separately (Fang et al., Nat. Struct. Biol. 6:1091-1095 (1999), which is hereby incorporated by reference in its entirety). The slow step in the folding of the catalytic domain involves consolidation of RNA structure around metal ions (Fang et al., Proc. Natl. Acad. Sci. USA 99:8518-8523 (2002), which is hereby incorporated by reference in its entirety). These observations suggest that short oligonucleotides could direct RNase P RNA into an inactive structure and thereby cause disruption of critical cellular processes that are dependent upon proper tRNA processing.

Internal ribosome entry sites (IRES) offer an alternative mode of ribosomal entry to an mRNA molecule (Hambidge and Sarnow, Proc. Natl. Acad. Sci. USA 89:10272-10276 (1992); Liebig et al., Biochem. 32:7581-7588 (1993), each of which is hereby incorporated by reference in its entirety). Normally, the entry of ribosomes into mRNA takes place via the cap located at the 5′ end of eukaryotic mRNAs. However, in some viral mRNAs the cap is absent and instead host ribosomes enter at an internal site of these mRNAs. To date, a number of these structures have been identified in the 5′ noncoding region of uncapped viral mRNAs, such as that, in particular, of picornaviruses, e.g., the poliomyelitis virus (Pelletier et al., Mol. Cell. Biol. 8:1103-1112 (1988), which is hereby incorporated by reference in its entirety) and the encephalomyocarditis virus (Jang et al., J. Virol. 62:2636-2643 (1988), which is hereby incorporated by reference in its entirety). The IRES is folded into a complex secondary structure and typically contains a pyrimidine-rich tract followed by an AUG codon (Agol, Adv. Virus Res. 40:103-180 (1991); Wimmer et al., Annu. Rev. Genet. 27:353-436 (1993); Sonennberg and Pelletier, BioEssays 11: 128-132 (1989), each of which is hereby incorporated by reference in its entirety). Internal ribosome entry has also been reported for other viral RNAs, including without limitation: hepatitis C virus (Lyons et al., J. Biol. Chem. E-publication M304052200v1.pdf at JBC on-line (2001), which is hereby incorporated by reference in its entirety), human herpesvirus type 8, which causes endothelial Kaposi's sarcoma (Low et al., J. Virol. 75:2938-2945 (2001), which is hereby incorporated by reference in its entirety), enteroviruses such as coxsakievirus and encephalomyocarditis virus (Rohll et al., J. Virol. 68:4384-4391(1994); Belsham, EMBO J. 11:1150-1110(1992), each of which is hereby incorporated by reference in its entirety), hepatitis A virus (Glass et al., Virology 193:842-852 (1993), which is hereby incorporated by reference in its entirety), and plautia stali intestine virus (Nishiyama et al., Nucleic Acids Res. 31:2434-2442 (2003), which is hereby incorporated by reference in its entirety).

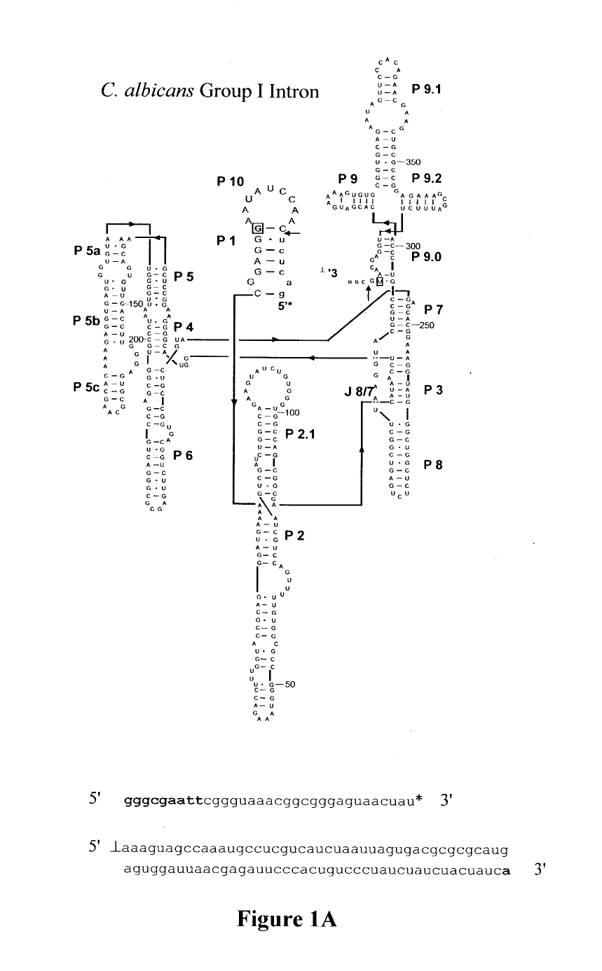

One preferred class of oligonucleotides of the present invention is directed to cause the misfolding of a Candida Group I self-splicing intron. In particular, this preferred class of oligonucleotides is characterized by binding to one or more of the following domains of the Candida Group I self-splicing intron: P2.1 domain, P3 domain, P6 domain, P7 domain, or P9.1 domain (see FIG. 1A). Exemplary oligonucleotides are those that possess the nucleotide sequence of TACCTTTC (SEQ ID NO: 13) or TCTACGACGGCC (SEQ ID NO: 14), wherein one or more of the bases is optionally modified by propynylation or alkylation, or one or more of the sugars is optionally locked to form an LNA base. These exemplary nucleotides include ^{L}(TACCTTTC) (SEQ ID NO: 5), T^{L}CT^{L}AC^{L}GA^{L}CG^{L}GC^{L}C (SEQ ID NO: 3) and a 2′-O-methyl derivative of TCTACGACGGCC (SEQ ID NO: 14).

Another preferred class of oligonucleotides of the present invention is directed to cause the misfolding of an E. coli RNase P RNA. In particular, this preferred class of oligonucleotides is characterized by binding to one or more of the following domains of the E. coli RNase P RNA: L8 domain, J 15/16 domain, or P4 domain. Exemplary oligonucleotides are those which possess the nucleotide sequence of CAGCCUACCCGG (SEQ ID NO: 12), wherein one or more of the bases is optionally modified by propynylation or alkylation, or one or more of the sugars is optionally locked to form an LNA base.

The oligonucleotides of the present invention can be readily developed to misfold a targeted RNA molecule. The development of such oligonucleotides can be achieved by predicting the folding structure of a target RNA molecule, designing the oligonucleotide to hybridize to the RNA molecule at a site critical for the secondary and/or tertiary folding thereof; and then determining whether binding of the oligonucleotide at the site modifies folding of the RNA molecule (i.e., prevents folding into the predicted folding structure or the active conformation) and thereby modifies the activity of the RNA molecule. By “critical” it is intended that the site bound by the oligonucleotide is either involved in tertiary structure formations (and it cannot become involved in forming such tertiary structure during folding of the RNA while the oligonucleotide is bound thereto) or binding of the oligonucleotide changes secondary folding structures to preclude the formation of other secondary and/or tertiary structures (due to intramolecular constraints).

To predict the folding structure of the target RNA molecule, any of a variety of suitable RNA folding software programs, e.g., RNAStructure program (available from D. Turner at the University of Rochester, Rochester, N.Y.), Mfold software package (available from M. Zucker at the Rensselear Polytechnic Institute, Rensselear, N.Y.), and Vienna RNA software package, including RNAfold, RNAeval, and RNAsubopt (available from I. Hofacker at the Institute for Theoretical Chemistry, Vienna Austria), can be used. Prediction of the folding structure of the target RNA molecule allows for the identification of domains that are likely to be required for secondary and/or tertiary folding of the RNA molecule into its active conformation. The lowest free energy state of the RNA molecule as predicted using such software may or may not be the true active conformation of the RNA molecule in a cellular environment. For example, the free energy state of RNA molecules possessing tertiary structures cannot be accurately predicted. It is believed, however, that more often than not the predicted lowest free energy state of the RNA molecule sufficiently resembles the true active conformation. By way of example, the RNAStructure program historically has, on average, predicted about 73 percent of canonical base pairs correctly (Mathews et al., J Mol. Biol. 288:911-940 (1999), which is hereby incorporated by reference in its entirety).

By identifying the domains that are likely to be required for secondary and/or tertiary folding of the RNA molecule, it is possible to identify oligonucleotides that will hybridize to those domains and therefore, using the RNA folding software programs, to predict whether those oligonucleotides will induce stable misfolding thereof. The predicted misfolded conformation is preferably characterized by a free energy state that is not more than the greater of either (a) about 10 percent or (b) about 10 kcal/mole higher than the lowest free energy state of the RNA molecule as predicted using the RNA folding software programs. The closer the free energy state of the predicted misfolded conformation to the predicted lowest free energy state of the RNA molecule, the more likely the predicted misfolded conformation will be stable. Generally, oligonucleotides with a high affinity for the binding site are preferred. For purposes of designing suitable oligonucleotides, therefore, selection of oligonucleotides that achieve misfolded structures (as described above) that are sufficiently close in free energy to the lowest predicted free energy state of the RNA molecule should allow for suitable modification of RNA activity as described above.

To determine whether the oligonucleotide causes misfolding of the RNA molecule, such misfolding can be predicted using suitable software of the type described above or by assessing physical modifications of RNA molecule behavior. The latter can be assessed, for example, by identifying altered mobility of the RNA molecule (relative to RNA not exposed to the oligonucleotide) during native gel electrophoresis or by exposing the RNA molecule to a suitable chemical modification agent in the presence or absence of the oligonucleotide and detecting modified nucleotides of the RNA molecule(s) to assess differential nucleotide modification (i.e., in the presence or absence of the oligonucleotide). Exemplary modification agents include, without limitation, diethyl pyrocarbonate (DEPC), dimethyl sulfate (DMS), kethoxal, 1-cyclohexyl-3-(2-morpholinoethyl)-carbodiimide metho-p-toluene-sulfonate (CMCT), hydroxyl radicals, nucleases, RNase H, and other modification enzymes. The design and preparation and the assessment of oligonucleotide efficacy can be carried out as described in greater detail in the accompanying examples (infra).

Oligonucleotides of the present invention can be prepared in vitro using known synthesis techniques or in vivo using cellular replicative machinery. Regardless of their mode of production or administration, the oligonucleotide directed misfolding of functional RNA molecules can afford a number of uses discussed below.

According to one aspect of the present invention, the RNA molecule whose function can be modified may be necessary to the survivability of a pathogen, in which case the oligonucleotides of the present invention can be used to disrupt the survivability of the pathogen. Pathogens whose survival can be affected include viruses, bacteria, yeast, and fungi. Exemplary pathogens that utilize particular types of functional RNA molecules are described above, without limitation to those listed. As a result of disrupting the activity of the functional RNA molecule (i.e., by binding the oligonucleotide to the RNA molecule), the disrupted activity of the RNA molecule results in disrupted survival of the pathogen. The pathogen whose survival is to be disrupted can be in vivo or ex vivo. By ex vivo it is intended that the pathogen can be located outside the body of another (higher) organism. By in vivo it is intended that the pathogen can be located inside the body of another (higher) organism. Disrupted survival can be manifested in any manner that decreases or substantially or completely inhibits the ability of the pathogen to reproduce itself or the ability of the pathogen to destroy a host cell.

Thus, a further aspect of the present invention relates to a method of treating or preventing pathogen infection in a patient. The patient is preferably a mammalian patient, although other higher organisms (such as reptiles, birds, marsupials, amphibians, and fish) can also be treated in accordance with the present invention. When treating or preventing pathogen infection in a patient, an oligonucleotide of the present invention is administered to the patient under conditions effective to cause uptake of the oligonucleotide by the pathogen or patient cells infected with the pathogen, whereby the oligonucleotide binds to the RNA molecule and the binding of the oligonucleotide causes the RNA molecule to misfold and adopt a conformation characterized by reduced activity, thereby disrupting survivability of the pathogen to treat or prevent infection of the patient by the pathogen.

According to another aspect of the invention, the RNA molecule whose function can be modified may be necessary to the survival of a cancer cell, in which case the oligonucleotides of the present invention can be used to disrupt the survival or proliferation of the cancer cells. For example, a number of cancers involve overexpression of mRNAs and the oligonucleotides of the present invention can be used to interfere with the activity or translation of overexpressed mRNAs. Exemplary mRNA molecules and associated cancers include, without limitation: a-Ras mRNA, which is implicated in numerous types of cancer (Downard, Nat. Rev. Cancer 3:11-22 (2003), which is hereby incorporated by reference in its entirety); cmyc mRNA, which is implicated in numerous types of cancers (Hermeking, Curr. Cancer Drug Targets 3:173-175 (2003), which is hereby incorporated by reference in its entirety); urokinase-type plasminogen activator mRNA, which is implicated in non-small cell lung cancer (Morita et al., Int. J Cancer 78:286-292 (1998), which is hereby incorporated by reference in its entirety); erB-2 (also known as HER-2 or neu), which is implicated in about one-third of all stomach, breast, and ovary cancers (Henry et al., Cancer Res. 53:1403-1408 (1993), which is hereby incorporated by reference in its entirety); and ATP-binding cassette transporter mRNA, which is implicated in lung cancer (Cole et al., Science 258:1650-1654 (1992), which is hereby incorporated by reference in its entirety). Other mRNAs whose overexpression is implicated in tumor development are continually being identified and can therefore be expected to be treated in accordance with the present invention. As a result of disrupting the activity of the functional RNA molecule (i.e., by binding the oligonucleotide to the RNA molecule), the disrupted activity of the RNA molecule results in disrupted survival or proliferation of the cancer cells in which the RNA molecule is overexpressed. The cancer cells whose survival or proliferation is to be disrupted can be in vivo or ex vivo. By ex vivo it is intended that the cancer cells be located outside the body, i.e., as a cell line. By in vivo it is intended that the cancer cells can be located inside the body of an organism, i.e., in the form of a tumor.

Thus, a further aspect of the present invention relates to a method of treating a cancerous or pre-cancerous condition in a patient. The patient is preferably a mammalian patient, although other higher organisms (such as reptiles, birds, marsupials, amphibians, and fish) can also be treated in accordance with the present invention. When treating a cancerous condition in a patient, an oligonucleotide of the present invention is administered to the patient under conditions effective to cause uptake of the oligonucleotide by the cancerous cells, whereby the oligonucleotide binds to the overexpressed mRNA molecule and the binding of the oligonucleotide causes the mRNA molecule to misfold and adopt a conformation characterized by reduced activity (i.e., translation) thereof, thereby disrupting survival or proliferation of the cancer cells to treat the cancerous condition. By treating the cancerous condition, it is intended that the cancerous condition can be brought into complete remission, the tumor size can be reduced (facilitating alternative treatment therapies in combination), or the growth rate of the tumor can be reduced (facilitating longer life-expectancy).

Regardless of its ultimate use, the oligonucleotide can be administered alone or in combination with pharmaceutically or physiologically acceptable carriers, excipients, or stabilizers, or in solid or liquid form such as, tablets, capsules, powders, solutions, suspensions, or emulsions, it can be administered orally, parenterally, subcutaneously, intravenously, intramuscularly, intraperitoneally, by intranasal instillation, by inhalation, by implantation, by intracavitary or intravesical instillation, intraocularly, intraarterially, intralesionally, transdermally, by application to mucous membranes (i.e., of the nose, throat, and bronchial tubes), rectally, vaginally, or topically. The mode of administration often dictates the desired formulation of the composition to be administered.

For injectable dosages, solutions or suspensions of these materials can be prepared in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the oligonucleotide in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

For parenteral administration, aqueous solutions of the oligonucleotide in water-soluble form can be used. Additionally, suspensions of the oligonucleotide may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran.

For rectal or vaginal delivery, the composition can be appropriately formulated as a suppository or retention enema, e.g., containing conventional suppository bases such as cocoa butter or other glycerides.

In addition to the formulations described previously, the oligonucleotide may also be formulated as a depot preparation. Such long acting formulations may be administered by implantation (for example subcutaneously or intramuscularly) or by intramuscular injection. Thus, for example, the oligonucleotide may be formulated with suitable polymeric or hydrophobic materials (for example as an emulsion in an acceptable oil) or ion exchange resins, or as sparingly soluble derivatives, for example, as a sparingly soluble salt. Selection of polymeric matrix material is based on biocompatibility, biodegradability, mechanical properties, cosmetic appearance and interface properties. The particular application of the oligonucleotide will define the appropriate formulation. Potential matrices for the compositions may be biodegradable and chemically defined calcium sulfate, tricalcium phosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate, as well as other materials that are known in the drug delivery arts. The bioceramics may be altered in composition, such as in calcium-aluminate-phosphate and processing to alter pore size, particle size, particle shape, and biodegradability.

The amount of oligonucleotide present in the pharmaceutical composition of the present invention will depend upon the nature and severity of the condition being treated, and on the nature of prior treatments which the patient has undergone. Ultimately, the attending physician will decide the amount of oligonucleotide with which to treat each individual patient. Initially, the attending physician will administer low doses of oligonucleotide and observe the patient's response. Larger doses of oligonucleotide may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 μg to about 10 mg, more preferably about 0.1 μg to about 1 mg) of oligonucleotide per kg body weight.

Oligonucleotides can be administered in the above formulations or as naked DNA-for cellular uptake. For instance, as described in copending U.S. Provisional Patent Application Serial No. 60/438,277, filed Jan. 3, 2003, which is hereby incorporated by reference in its entirety, oligonucleotides are selectively taken up by Candida and Saccharomyces at higher rates than by patient cells. In accordance with the disclosure therein, it should be noted that the pH of the environment in which the Candida or Saccharomyces exist can play a role in modifying the selectivity of uptake by the Candida or Saccharomyces relative to the patient cells. It is preferable that the pH is at physiological levels. In certain environments within the mammalian body, physiological levels can deviate significantly from neutral pH (˜7). Thus, in certain aspects of the present invention the environment can have a pH less than or equal to about 4.5 or, more particularly, between about 2 to about 4.5.

Alternatively, a colloidal dispersion system can be used to deliver the oligonucleotide to the patient for targeted delivery. Colloidal dispersion systems include macromolecule complexes, nanocapsules, microspheres, beads, and lipidbased systems including oil-in-water emulsions, micelles, mixed micelles, and liposomes. The preferred colloidal system of this invention is a lipid preparation including unilamaller and multilamellar liposomes.

Liposomes are artificial membrane vesicles that are useful as delivery vehicles in vitro and in vivo. It has been shown that large unilamellar vesicles (LUV), which range in size from about 0.2 to about 4.0 μm, can encapsulate a substantial percentage of an aqueous buffer containing the oligonucleotides of the present invention (Fraley et al., Trends Biochem. Sci. 6:77 (1981), which is hereby incorporated by reference in its entirety). In addition to mammalian cells, liposomes have been used for delivery of polynucleotides in yeast and bacterial cells. In order for a liposome to be an efficient transfer vehicle, the following characteristics should be present: (1) encapsulation of the oligonucleotides at high efficiency while not compromising their biological activity; (2) preferential and substantial binding to a target cell in comparison to non-target cells; (3) delivery of the aqueous contents of the vesicle to the target cell cytoplasm at high efficiency; and, optionally, (4) accurate and effective expression of genetic information (Mannino et al., Biotechniques 6:682 (1988), which is hereby incorporated by reference in its entirety). In addition to such LUV structures, multilamellar and small unilamellar lipid preparations which incorporate various cationic lipid amphiphiles can also be mixed with anionic oligonucleotides to form liposomes (Felgner et al., Proc. Natl. Acad. Sci. USA 84(21): 7413 (1987), which is hereby incorporated by reference in its entirety).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. The appropriate composition and preparation of cationic lipid amphiphile:oligonucleotide formulations are known to those skilled in the art, and a number of references which provide this information are available (Bennett et al., J. Liposome Research 6(3):545 (1996), which is hereby incorporated by reference in its entirety).

Examples of lipids useful in liposome production include phosphatidyl compounds, such as pbosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine. Examples of cationic amphiphilic lipids useful in formulation of nucleolipid particles for polynucleotide delivery include the monovalent lipids N-[1-(2,3-dioleoyloxy)propyl]-N,N,N,-trimethyl ammonium methyl-sulfate, N-[2,3-dioleoyloxy)propyl]-N,N,N-trimethyl ammonium chloride, and DC-cholesterol, the polyvalent lipids LipofectAMINE™, dioctadecylamidoglycyl spermine, Transfectam™, and other amphiphilic polyamines. These agents may be prepared with helper lipids such as dioleoyl phosphatidyl ethanolamine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization. The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

As an alternative to the use of liposomal delivery systems or for use in combination therewith, viral vectors can be used to transform target cells so that they express the oligonucleotide in the target cell, thereby modifying activity of RNA molecules in the same target cell.

When a vector is employed to express an oligonucleotide of the present invention, such as an oligoRNA, in a target cell, conventional recombinant techniques can be employed to prepare a DNA construct that encodes the oligonucleotide and ligate the same into the viral vector (Sambrook et al., Molecular Cloning: A Laboratory Manual, Second Edition, Cold Spring Harbor Press, NY (1989), which is hereby incorporated by reference in its entirety). The viral vector so prepared can be maintained ex vivo in appropriate host cell lines, which may include bacteria, yeast, mammalian cells, insect cells, plant cells, etc. For example, having identified the oligoRNA to be expressed in the target cell, a DNA molecule that encodes the oligoRNA can be ligated to appropriate 5′ promoter regions and 3′ transcription termination regions, forming a DNA construct, so that the oligoRNA will be appropriately expressed in transformed target cells (Sambrook et al., Molecular Cloning: A Laboratory Manual, Second Edition, Cold Spring Harbor Press, NY (1989), which is hereby incorporated by reference in its entirety). The selection of appropriate 5′ promoters and 3′ transcription termination regions is well known in the art and can be performed with routine skill. Suitable promoters for use in mammalian cells include, without limitation, SV40, MMTV, metallothionein-l, adenovirus Ela, CMV, immediate early, immunoglobulin heavy chain promoter and enhancer, and RSV-LTR.

Any suitable viral vector can be utilized to express an oligonucleotide of the present invention. When transforming mammalian cells for heterologous expression of an oligonucleotide of the present invention, exemplary viral vectors include adenovirus vectors and retroviral vectors. Other suitable viral vectors now known or hereafter developed can also be utilized to deliver the oligonucleotide of the present invention into cells.

Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in its entirety. Adeno-associated viral gene delivery vehicles can be constructed and used to deliver a gene to cells. The use of adeno-associated viral gene delivery vehicles in vitro is described in Chatterjee et al., Science 258:1485-1488 (1992); Walsh et al., Proc. Nat'l Acad. Sci. USA 89:7257-7261 (1992); Walsh et al., J. Clin. Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:12611267 (1995); and Zhou et al., Gene Ther. 3:223-229 (1996), each of which is hereby incorporated by reference in its entirety. In vivo use of these vehicles is described in Flotte et al., Proc. Nat'l Acad. Sci. USA 90:10613-10617 (1993); and Kaplitt et al., Nature Genet. 8:148-153 (1994), each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, each of which is hereby incorporated by reference in its entirety).

Retroviral vectors which have been modified to form infective transformation systems can also be used to deliver nucleic acid encoding a desired oligonucleotide of the invention into a target cell. One such type of retroviral vector is disclosed in U.S. Pat. No. 5,849,586 to Kriegler et al., which is hereby incorporated by reference in its entirety.

Regardless of the type of infective transformation system employed, it can be targeted for delivery of the oligonucleotide to a specific cell type. For example, for delivery of the oligonucleotide into tumor cells, a high titer of the infective transformation system can be injected directly within the tumor site so as to enhance the likelihood of tumor cell infection. The infected cells will then express the desired oligonucleotide to disrupt either the survival or the proliferation of the cancer cell.

As another alternative to liposomal delivery or infective transformation vectors, oligonucleotide-polypeptide conjugates can be utilized. Any suitable oligonucleotide-polypeptide conjugate that is capable of delivering the oligonucleotide into a target cell and to the targeted RNA molecule can be employed in conjunction with the present invention. One exemplary oligonucleotide-polypeptide conjugate, which includes an oligonucleotide complexed to a conjugate of a cell-specific binding agent and an oligonucleotide binding agent, is described in U.S. Pat. No. 6,030,954 to Wu et al., which is hereby incorporated by reference in its entirety. Another exemplary oligonucleotide-polypeptide conjugate, which includes an oligonucleotide covalently linked to a peptide (which is capable of being cleaved by proteolytic enzymes inside the target cell) that in turn is covalently linked to a carrier or targeting ligand moiety (which facilitates delivery of the entire conjugate into a target cell), is described in U.S. Pat. No. 5,574,142 to Meyer, Jr., et al., which is hereby incorporated by reference in its entirety. Other oligonucleotide-polypeptide conjugates now known or hereafter developed can also be utilized to deliver the oligonucleotides of the present invention into cells.

The oligonucleotides of the present invention, or compositions containing the same, can also be administered in combination with other known therapeutic or preventative agents for purposes of treating or preventing a disease, disorder, pathogen infection, or cancerous condition as described herein. Therapeutic and preventative agents are known and continually being developed for treating or preventing various pathogen infections, diseases, disorders, or cancerous conditions.

### EXAMPLES

The following Examples are intended to be illustrative and in no way are intended to limit the scope of the present invention.

Materials and Methods

Buffers: Transcription buffer used in Example 1 contains 40 mM Tris-HCl (pH 7.5), 62.5 ng/μL BSA, 5 mM spermidine, 5 mM dithiothreitol, 14 mM MgCl₂, 1 mM each nucleotide triphosphate, 3 ng linearized C-h plasmid (Disney et al., Biochemistry 40:6507-6519 (2001), which is hereby incorporated by reference in its entirety) that contains precursor sequence, α-ATP (30 Ci/mmol), and 50 U T7 RNA polymerase (New England BioLabs). Transcription buffer used in Example 2 contains 1×H14Mg, 1 mM each NTP, 2 mM DTT, and 2 mM spermidine. HXMg is 50 mM Hepes (pH 7.5) (25 mM Na⁺), 135 mM KCl, and X mM MgCl₂ (e.g., HOMg contains no MgCl₂ and H10Mg contains 10 mM MgCl₂).

Oligonucleotides: Oligonucleotides were synthesized, deblocked, and purified by standard methods (Wincott et al., Nucleic Acids Res. 23:2677-2684 (1995); Caruthers et al., Methods Enzymol. 211:3-20 (1992); Matteucci et al., Tetrahedron Lett. 21:719722 (1980); Xia et al., Biochemistry 37:14719-14735 (1998), each of which is hereby incorporated by reference in its entirety). Concentrations were determined from predicted extinction coefficients and measured absorbances at 260 or 280 nm at 25° C. (Puglisi et al., Methods Enzymol. 180:304-325 (1989), which is hereby incorporated by reference in its entirety). Some oligonucleotides were also characterized by MS with a Hewlett Packard 1100 LC/MS Chemstation. Locked Nucleic Acids (LNAs) were purchased from Proligo LLC and purified by reverse phase chromatography. Masses were confirmed by MALDI-MS. For LNA/DNA chimeras, LNA residues are denoted with ^{L}(e.g., ^{L}A) while DNA residues are represented only by their bases (e.g., A).

Optical Melting Experiments: Optical melting experiments were completed in 20 mM sodium cacodylate, 0.1 mM NaCl, and 0.5 mM EDTA, pH 7. Low NaCl concentration was used because melting temperatures for some duplexes were too high to measure at higher NaCl concentrations. An equal amount of each strand was mixed in buffer and a temperature gradient from 0 to 90° C. was applied. The resulting absorbance versus temperature curves were analyzed with MeltWin® (McDowell et al., Biochemistry 35:14077-14089 (1996), which is hereby incorporated by reference in its entirety). For each sequence, at least five different concentrations were analyzed over at least a 10-fold concentration range.

Oligonucleotide Screen and Dose Response Curves (Example 1): Oligonucleotides were initially screened by transcribing the precursor in the presence of 100 μM deoxyoligonucleotide for 1 h at 37° C. Transcription products were separated on 5% polyacrylamide denaturing gels. Results were imaged on a phosphorimager as described (Disney et al., Biochemistry 40:6507-6519 (2001), which is hereby incorporated by reference in its entirety). Dose response curves were then measured for oligonucleotides that limited splicing to 20% or less. Each dose response curve is an average of at least two assays, reported with standard error. IC50's were determined by fitting dose response curves with SigmaPlot® 2001's Logistic, 4 Parameter curve fit. Dose response curves were also measured in the presence of up to 11 mM bulk RNA from Torula Yeast (Sigma). The concentration of bulk RNA was estimated with an extinction coefficient at 260 nm of 10405 M⁻¹cm⁻¹nt⁻¹ obtained by averaging the extinction coefficients for dinucleotides (Puglisi et al., Methods Enzymol. 180:304-325 (1989), which is hereby incorporated by reference in its entirety).

Oligonucleotide Screen and Dose Response Curves (Example 2): Transcription reactions were run in 20 μL containing 1× transcription buffer, 50 units T7 RNA polymerase, 100 ng linearized RNase P RNA plasmid, 5 ng of RNase P protein, and trace internally labeled pre-tRNA annealed as above. Transcription mixtures with and without ODMiR oligonucleotides were incubated for 1 h at 37° C., and then ethanol precipitated. Products were separated on an 8% polyacrylamide denaturing gel. Results were quantified with a phosphorimager and ImageQuant v5.2. IC₅₀'s were obtained by fitting dose response curves to a 4-parameter logistic curve (SigmaPlot2001).

Native Gel Electrophoresis: Internally labeled C. albicans ribozyme (Disney et al., Biochemistry 40:6507-6519 (2001), which is hereby incorporated by reference in its entirety) was purified on a 5% polyacrylamide denaturing gel. The RNA was extracted from the gel by the crush and soak method, 2-butanol concentrated, and ethanol precipitated. Effects of oligonucleotides on folding were assayed by annealing C. albicans ribozyme and 1 [M oligonucleotides in HOMg buffer at 68° C. for 5 min, followed by slow cooling to 37° C. MgCl₂ was added to a final concentration of 10 mM and the samples allowed to equilibrate at 37° C. for 30 min. The samples were placed on ice, and loaded on a 7% polyacrylamide native gel containing H10Mg buffer, which was also used as the running buffer.

Diethyl Pyrocarbonate Modification (Example 1): The C. albicans ribozyme, 2 μM GACUCU (SEQ ID NO: 8) (a mimic of its native substrate), and oligonucleotides were annealed in HOMg buffer at 68° C. for 5 min. The samples were slow cooled to 37° C. MgCl₂ was added to a final concentration of 10 mM, and the samples were then incubated at 37° C. for 30 min. Diethyl pyrocarbonate (DEPC) was added to a final concentration of 650 mM and samples were incubated for 20 min at 37° C. (Banerjee et al., Biochemistry 34:6504-6512 (1995), which is hereby incorporated by reference in its entirety). The reactions were quenched by ethanol precipitation. Sites of modification were detected by primer extension using AMV Reverse Transcriptase (Life Science, Inc.) according to manufacturer's protocol except that samples were annealed in 435 mM NaOOCCH₃ instead of water. The ribozyme was sequenced by the Sanger method with reverse transcriptase (Sanger et al., Proc. Natl. Acad. Sci. USA 74:5436-5467 (1977), which is hereby incorporated by reference in its entirety).

Diethyl Pyrocarbonate Modification (Example 2): Structural changes due to ODMiR oligonucleotides were probed with DEPC by annealing 44 nM RNase P RNA, 220 nM pre-tRNA with or without 2 μM m(CAGCCUACCCGG) (SEQ ID NO: 12) at 90° C. for 3 min, followed by slow cooling to 37° C. MgCl₂ was added to 10 mM, and the samples incubated for 1 h at 37° C. The samples were incubated with 650 mM DEPC for 20 min at 37° C., and the reactions quenched by addition of 2.5 volumes of ethanol (Banerjee et al., Biochemistry 34:6504-6512 (1995), which is hereby incorporated by reference in its entirety). RNase P RNA was ethanol precipitated, and reverse transcribed to detect sites of increased modification. RNase P RNA was sequenced by the Sanger method with reverse transcriptase (Sanger et al., Proc. Natl Acad. Sci. USA 74:5436-5467 (1977), which is hereby incorporated by reference in its entirety). Band intensities were normalized separately to residue C50 and to full length product. Structural changes for nucleotides were reported if there was at least a 20% increase in modification for both normalizations. The same changes were observed in the presence or absence of pre-tRNA.

OligoWalk™ Predictions: The ΔG^{o}₃₇'s of duplex formation between all possible 12mer oligonucleotides and RNase P RNA were calculated by the OligoWalk™ program (Mathews et al., RNA 5:1458-1469 (1999), which is hereby incorporated by reference in its entirety) with the following settings: refold RNA, include suboptimal structures (from the RNAStructure program (Mathews et al., J. Mol. Biol. 288:911 940 (1999), which is hereby incorporated by reference in its entirety)), and DNA or RNA (approximation for 2′-O-Me RNA) oligonucleotides at 1 μM concentration.

RNA and Protein Clones: Plasmids containing the E. coli RNase P RNA (M1) gene (pDW98) and RNase P protein (C5) gene (PECPE1), and the B. subtilis pre-tRNA^{Asp} gene (PDW152) (Beebe et al., Biochemistry 33:10294-304 (1994); Zito et al., Nucleic Acids Res. 21:5916-5920 (1993), each of which is hereby incorporated by reference in its entirety) were generously supplied by Professor Norman R. Pace.

Preparation of E. coli RNase P Protein: RNase P protein was expressed and purified by the method of A. W. Poole and N. R. Pace (personal communication). The RNase P protein plasmid was transformed into BL21 (DE3) pLysS cells for expression. Protein expression was induced by addition of 1 mM IPTG and incubation for 4 h at 37° C. Cells were lysed by sonication in 50 mM Tris-HCl, pH 8.0, 5 mM EDTA, 100 mM DTT, and 10% glycerol. Lysate was brought to final concentrations of 5 M urea, 50 mM NaCl, 50 mM Tris-HCl, pH 8.0, and 1 mM DTT and purified by an SP Sepharose column (Amersham) using a linear gradient from 50 mM to 2 M NaCl. Fractions containing RNase P protein were pooled and dialyzed into 5 M urea, 50 mM NaCl, 50 mM Tris-HCl, pH 7.5, and 1 mM DTT. The dialyzed fractions were loaded on a pre-packed Mono-S column (Amersham) with a linear gradient from 50 mM to 2 M NaCl. Fractions containing RNase P protein were dialyzed into 1× TE, pH 7.5, 1 mM DTT, and 50% glycerol, and concentrated using a YM-10 cartridge (Centricon). RNase P protein was then passed through a 0.2 μm microfuge tube filter (Corning).

Preparation of pre-tRNA: B. subtilis pre-tRNA^{Asp} was prepared by run-off transcription with T7 RNA polymerase (New England BioLabs) from its linearized plasmid. The pre-tRNA was purified on an 8% polyacrylamide denaturing gel, and electroeluted in 1× TBE. Purified pre-tRNA was desalted by a Sephadex column.

Internally labeled material was prepared with [α-³²P]ATP, and purified on an 8% polyacrylamide denaturing gel; the full-length product was excised from the gel, and eluted by the crush and soak method. Internally labeled pre-tRNA was then concentrated with 2-butanol, and ethanol precipitated. For each assay, pre-tRNA was annealed in 1× H0Mg buffer for 3 min at 90° C., followed by slow cooling to 37° C., at which point MgCl₂ was added to a final concentration of 10 mM.

### Example 1

### Oligonucleotide Directed Misfolding of C. albicans Group I Intron

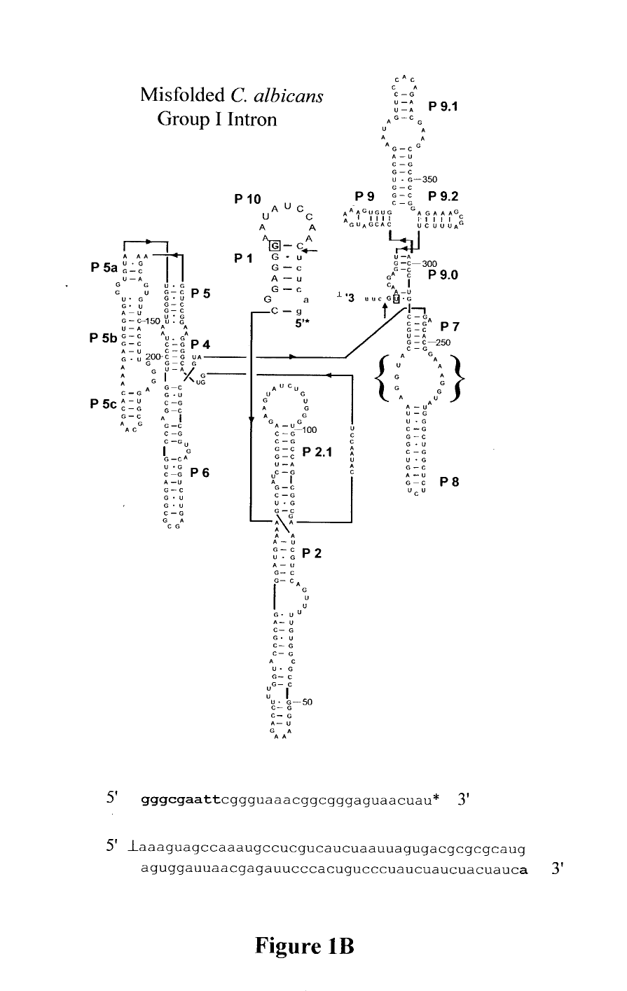

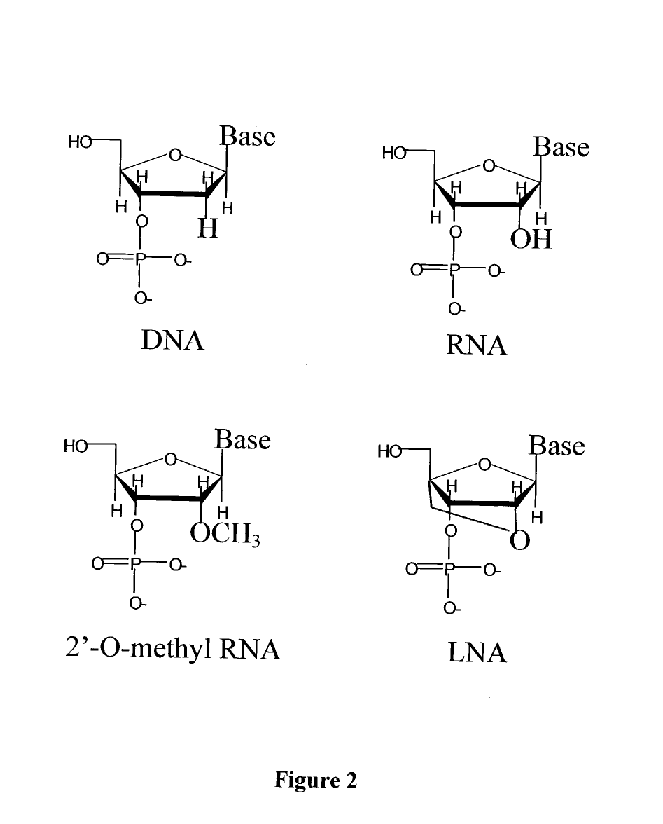

FIG. 1A shows the functional secondary structure of the C. albicans group I intron and FIG. 1B shows a suboptimal structure predicted by the program RNAStructure (Mathews et al., J. Mol. Biol. 288:911-940 (1999), which is hereby incorporated by reference in its entirety). The suboptimal structure is only 2.2 kcal/mol less stable than the predicted lowest free energy structure, and differs from the functional structure by replacement of P3 with a 5×8 nucleotide internal loop. P3 is part of a pseudoknot that is a rate limiting step in folding of the full length group I intron from T. thermophila (Banerjee et al., Biochemistry 34:6504-6512 (1995); Zarrinkar et al., Science 265:918-924 (1994); Sclavi et al., Science 279:1940-1943 (1998), each of which is hereby incorporated by reference in its entirety). This suggested that oligonucleotides complementary to nucleotides 252-259 in the internal loop could stabilize this misfold during transcription and thus inhibit self-splicing. This was tested by including ^{L}(TACCTTTC) (SEQ ID NO: 5), where L denotes LNA® nucleotides (see FIG. 2) in a transcription mixture. As seen in FIG. 3, ^{L}(TACCTTTC) (SEQ ID NO: 5) inhibits 50% of self-splicing at 150 nM. Equivalent sequence oligonucleotides with DNA and RNA backbones did not inhibit self-splicing at 100 μM, while propynylation of the C5 position of pyrimidines in a DNA oligonucleotide inhibited self-splicing only at concentrations greater than 3 μM. Two control molecules, ^{L}(CCTTATCT) (SEQ ID NO: 6) and ^{L}(ACTCACCT) (SEQ ID NO: 7), decrease splicing only at concentrations greater than 10 μM. ^{L}(CCTTATCT) (SEQ ID NO: 6) has the same base composition as the ODMiR oligonucleotide, ^{L}(TACCTTTC) (SEQ ID NO: 5), but is not complementary to any region of the intron. ^{L}(ACTCACCT) (SEQ ID NO: 7) is complementary to nucleotides 176-183 which are base paired in helix P5b. To test the specificity of ^{L}(TACCTTTC) (SEQ ID NO: 5) for the group I intron, Torula Yeast bulk RNA was added to transcription mixtures. Nucleotide concentrations up to II mM (˜25,000 times the nucleotide concentration of the group I intron) did not significantly affect the IC50 of ^{L}(TACCTTTC) (SEQ ID NO: 5) for inhibiting group I intron splicing.

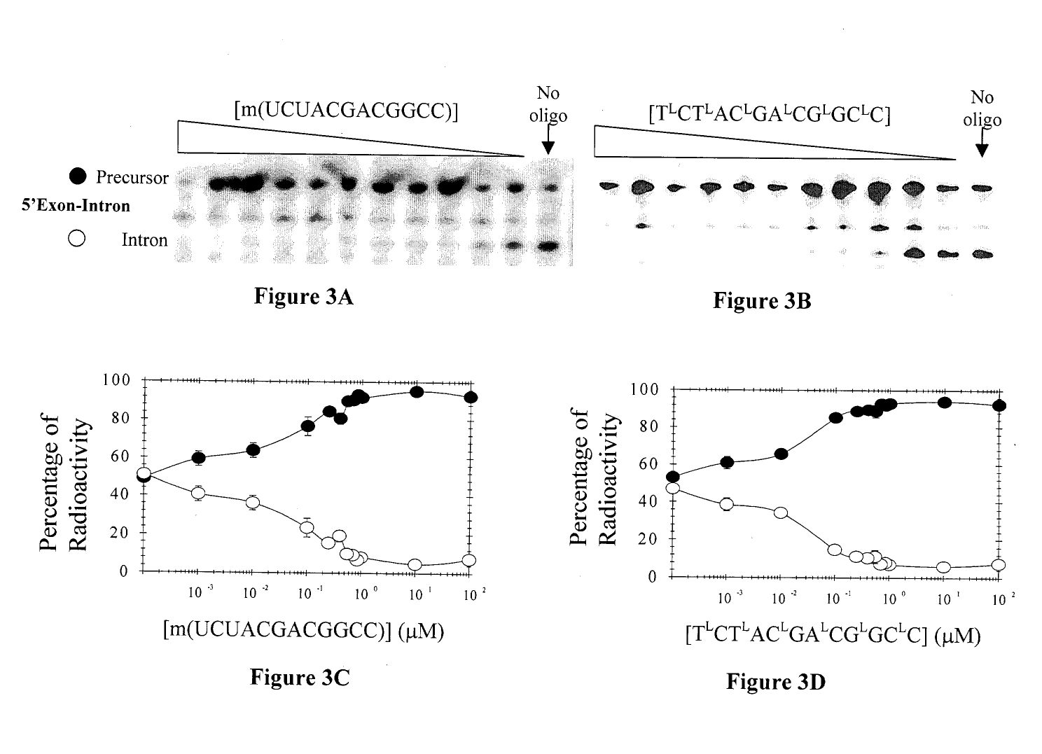

As an alternative to rational design, a library of 33 deoxyoligonucleotides of consecutive 12-mers complementary to the C. albicans group I intron's primary sequence was screened in the transcription assay. At 100 μM concentration, most sequences had little or no effect on splicing. However, d(TCTACGACGGCC) (SEQ ID NO: 14), which is complementary to nucleotides 235-246, has an IC50 of 1 μM. Increasing the length of this oligonucleotide by 3 nucleotides in either the 5′ or 3′ directions did not improve the IC50. Shifting the complementarity of the oligonucleotide by 6 nucleotides to the 5′ or 3′ direction in the intron also did not improve the IC50. In order to improve the IC50 of this molecule, modifications were made to sugar moieties to give 2′-O-methyl oligonucleotides and LNAs (FIG. 2). The 2′-O-methyl analog improved the IC50 to about 50 nM, while the oligonucleotide with alternating deoxy and locked sugars, T^{L}CT^{L}AC^{L}GA^{L}CG^{L}GC^{L}C (SEQ ID NO: 3), gave an IC50 of about 30 nM (FIG. 4). The control sequence A^{L}CT^{L}CG^{L}CA^{L}GT^{L}CG^{L}C (SEQ ID NO: 4), which has the same base composition, inhibits self-splicing only at concentrations >10 μM. Addition of up to 11 mM bulk Torula Yeast RNA did not significantly affect the IC50 of T^{L}CT^{L}AC^{L}GA^{L}CG^{L}GC^{L}C (SEQ ID NO: 3) for inhibition of self-splicing.

Binding sites for ^{L}(TACCTTTC) (SEQ ID NO: 5) and T^{L}CT^{L}AC^{L}GA^{L}CG^{L}GC^{L}C (SEQ ID NO: 3) were determined by reverse transcription stops in the presence and absence of oligonucleotide. Stops are observed at binding sites because reverse transcriptase is unable to proceed through the oligonucleotide. ^{L}(TACCTTTC) (SEQ ID NO: 5) and T^{L}CT^{L}AC^{L}GA^{L}CG^{L}GC^{L}C (SEQ ID NO: 3) bind as designed to nucleotides 176-183 and 235-246, respectively.

The strength of base pairing between several oligonucleotides and their RNA complements was measured by optical melting, the results of which are illustrated in Tables 1 and 2 below. As shown in Table 1, stronger base pairing usually provides a lower IC50. The exception is T^{L}CT^{L}AC^{L}GA^{L}CG^{L}GC^{L}C (SEQ ID NO: 3), which has an IC50 similar to its 2′-O-methyl equivalent even though its base pairing is more favorable by 7 kcal/mol at 37° C.

TABLE 1

IC50s for Inhibition of Self-splicing by ODMiR
Oligonucleotides and Their Affinities for Binding
to a Complementary RNA

| | | |
|---|---|---|
| | −ΔG°₃₇ | IC50 |
| Oligonucleotide Sequence | (kcal/mol) | (μM) |

| | | |
|---|---|---|
| 5′d(TCTACGACGGCC) | 8.9 ± 0.2^{a} | 1 |
| (SEQ ID NO: 14) | | |
| 5′m(UCUACGACGGCC) | 14.7 ± 0.3^{a} | 0.05 |
| (SEQ ID NO: 12) | | |
| 5′T^{L}CT^{L}AC^{L}GA^{L}CG^{L}GC^{L}C | 21.8 ± 3.4^{a} | 0.03 |
| (SEQ ID NO: 3) | | |
| 5′r(UACCUUUC) | 4.9 ± 0.6^{b} | >100 |
| (SEQ ID NO:15) | | |
| 5′d(^{P}UA^{P}C^{P}C^{P}U^{P}U^{P}U^{P}C) | 9.1 ± 0.1^{b} | 4 |
| (SEQ ID NO:10) | | |
| 5′^{L}(TACCTTTC) | 14.0 ± 0.8^{b} | 0.15 |
| (SEQ ID NO: 5) | | |

The melting curves were measured for binding to
^{a}3′r(AGAUGCUGCCGG) (SEQ ID NO: 16) or
^{b}3′r(AUGGAAAG) (SEQ ID NO: 17) in 20 mM sodium cacodylate, 0.1 mM NaCl, and 0.5 mM EDTA, pH 7

TABLE 2

Thermodynamic Parameters for Binding of Modified Oligonucleotides to their
Complementary Sequence in the Group I Intron

| | |
|---|---|
| T_{M} Dependence | Curve Fitting |

| | | | | | | | | |
|---|---|---|---|---|---|---|---|---|
| | −ΔG°₃₇ | −ΔH° | −ΔS° | T_{M} | −ΔG°₃₇ | −ΔH° | −ΔS° | T_{M} |
| Sequence | (kcal/mol) | (kcal/mol) | (eu) | (° C.) | (kcal/mol) | (kcal/mol) | (eu) | (° C.) |

| | | | | | | | | |
|---|---|---|---|---|---|---|---|---|
| d(TCTACGACGGCC) | 8.9 ± | 75.4 ± | 214.3 ± | 47.2 | 9.1 ± | 81.4 ± | 232.9 ± | 47.2 |
| (SEQ ID NO:14) | 0.15^{a} | 5.17 | 16.27 | | 0.37 | 9.42 | 29.30 | |
| m(UCUACGACGGCC) | 14.7 ± | 94.5 ± | 257.1 ± | 66.4 | 15.8 ± | 108.7 ± | 299.5 ± | 66 |
| (SEQ ID NO:12) | 0.26^{a} | 3.34 | 9.92 | | 0.36 | 3.66 | 10.76 | |
| T^{L}CT^{L}AC^{L}GA^{L}CG^{L}GC^{L}C | 21.8 ± | 123.3 ± | 327.2 ± | 80.9 | 20.9 ± | 116.2 ± | 307.1 ± | 80.9 |
| (SEQ ID NO:3) | 3.40^{a} | 31.15 | 88.14 | | 3.26 | 28.12 | 80.39 | |
| r(UACCUUUC) | 4.9 ± | 73.1 ± | 219.8 ± | 30.2 | 4.4 ± | 94.4 ± | 290.0 ± | 30.2 |
| (SEQ ID NO:15) | 0.59^{b} | 15.55 | 50.91 | | 0.35 | 10.56 | 34.68 | |
| d(^{P}UA^{P}C^{P}C^{P}U^{P}U^{P}U^{P}C) | 9.1 ± | 56.5 ± | 153.0 ± | 51.8 | 9.8 ± | 75.0 ± | 210.3 ± | 51 |
| (SEQ ID NO:10) | 0.02^{b} | 0.74 | 2.29 | | 0.19 | 3.38 | 11.30 | |
| ^{L}(TACCTTTC) | 14.0 ± | 81.3 ± | 217.1 ± | 67.8 | 14.9 ± | 92.5 ± | 250.3 ± | 68.3 |
| (SEQ ID NO:5) | 0.75^{b} | 8.52 | 25.18 | | 1.08 | 12.11 | 35.75 | |

The melting curves were measured for binding to
^{a}3′r(AGAUGCUGCCGG) (SEQ ID NO:16) or
^{b}3′r(AUGGAAAG) (SEQ ID NO: 17) in 20 mM sodium cacodylate, 0.1 mM NaCl, and 0.5 mM EDTA, pH 7.
Here, d denotes deoxyribonucleic acid, m, 2′O-methyl; L, locked nucleic acid; r, ribonucleic acid, and P, C5-1-propynyl.
T_{M}is reported for 100 μM total strand concentration.

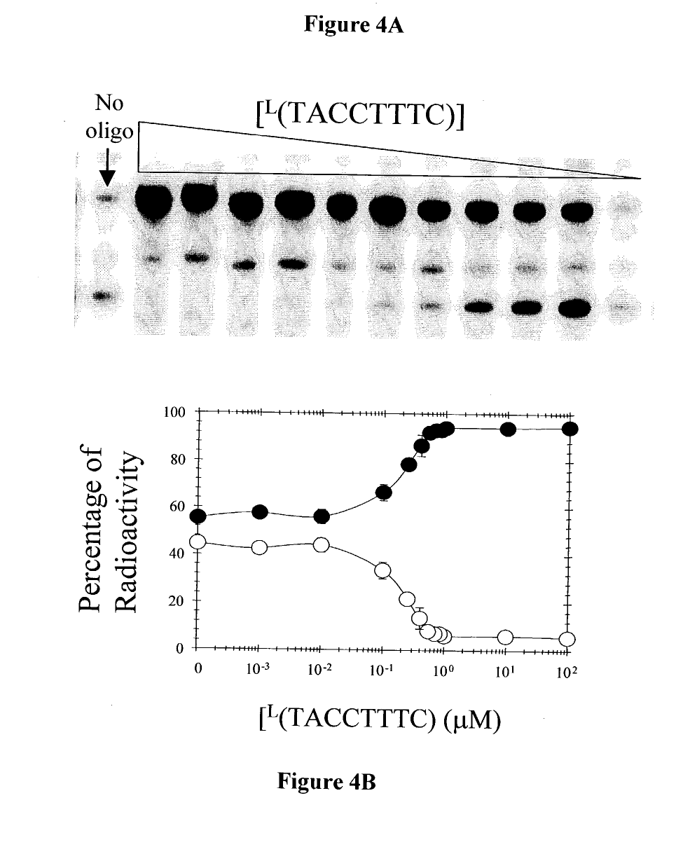

To further test the ability of oligonucleotides to stabilize misfolds, the ribozyme was reannealed in the presence of m(UCUACGACGGCC) (SEQ ID NO: 12), T^{L}CT^{L}AC^{L}GA^{L}CG^{L}GC^{L}C (SEQ ID NO: 3), and ^{L}(TACCTTTC) (SEQ ID NO: 5), and analyzed by native gel electrophoresis. As shown in FIG. 5, the oligonucleotides decrease mobility through the gel as compared to ribozyme reannealed in the absence of oligonucleotide or in the presence of substrate analogs—r(GACUCU) (SEQ ID NO: 8) and r(U₆GACUCU) (SEQ ID NO: 9), which bind tightly to the ribozyme (Disney et al., Biochemistry 40:6507-6519 (2001), which is hereby incorporated by reference in its entirety). U₆GACUCU (SEQ ID NO: 9) provides a control for the effect of added charge because the total net charge is the same for this molecule as for the longest oligonucleotide tested. Control molecules that contain the same base composition as oligonucleotides that inhibit splicing or that target a site that is completely paired in the native secondary structure, however, do not show reduced mobility (FIG. 5). This suggests that molecules that inhibit splicing are misfolding the intron.

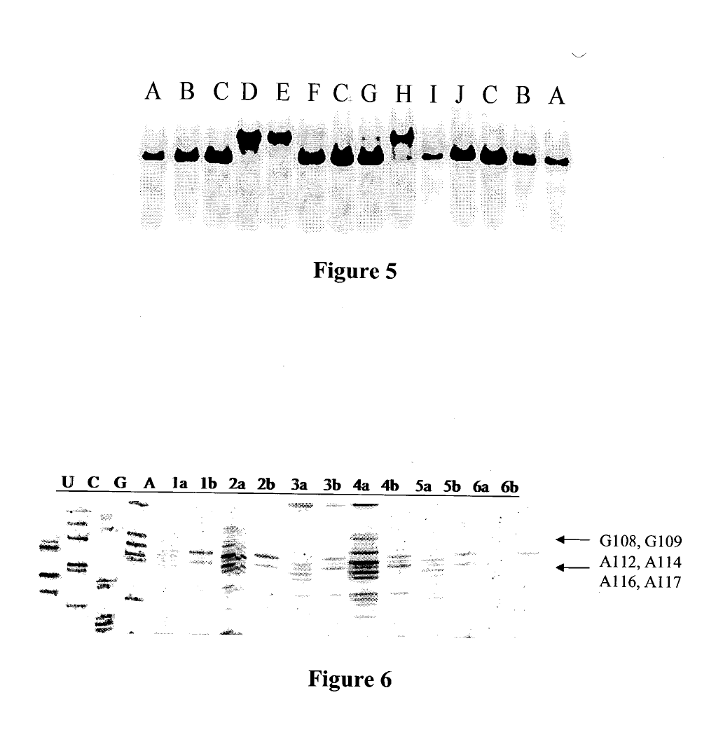

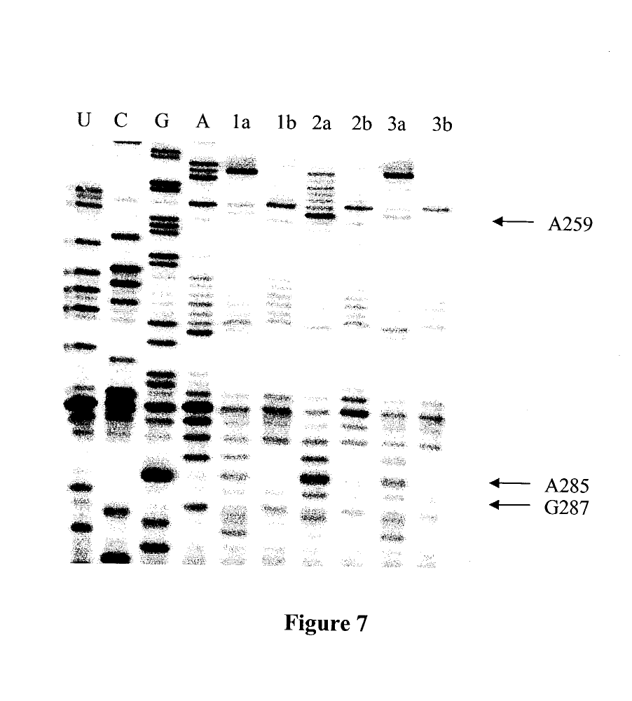

Diethyl pyrocarbonate was used to probe for changes in the structure of the ribozyme when reannealed in the presence of r(GACUCU) (SEQ ID NO: 8) and in the absence or presence of oligonucleotides. DEPC modifies the N7 position of A's and G's, thus giving insight into tertiary structure (Peattie et al., Proc. Natl. Acad. Sci. USA 77:4679-4682 (1980); Ehresmann et al., Nucleic Acids Res. 15:91099128 (1987), each of which is hereby incorporated by reference in its entirety). Sites of modification were detected by reverse transcription. When ribozyme was reannealed with r(GACUCU) (SEQ ID NO: 8) and either T^{L}CT^{L}AC^{L}GA^{L}CG^{L}GC^{L}C (SEQ ID NO: 3) or ^{L}(TACCTTTC) (SEQ ID NO: 5), increased modification of N7s is seen at G98-99, G108-109, A112, A114, and A116-A117, when compared to ribozyme reannealed only with r(GACUCU) (SEQ ID NO: 8) (see FIG. 6). Additional sites of increased modification are seen when ribozyme is annealed with r(GACUCU) (SEQ ID NO: 8) and T^{L}CT^{L}AC^{L}GA^{L}CG^{L}GC^{L}C (SEQ ID NO: 3). These include A259, A285, and G287 (FIG. 7).

RNA is an emerging target for therapeutics, and it is likely that there are many ways of inhibiting RNA function. Oligonucleotides are an attractive class of molecules for targeting RNA because much is known about the principles of molecular recognition between oligonucleotides and RNA. Moreover, oligonucleotide analogs are readily available (Freier et al., Nucleic Acids Res. 25:4429-4443 (1997), which is hereby incorporated by reference in its entirety). This facilitates rational design and screening of inhibitors. Moreover, the pharmacokinetic properties of oligonucleotides are relatively independent of sequence (Crooke et al., J. Pharmacol. Exp. Ther. 277:923-937 (1996), which is hereby incorporated by reference in its entirety) which should further simplify the drug discovery process. Here, we demonstrate that oligonucleotide directed misfolding of RNA provides an approach that can be used to target RNA with short oligonucleotides.

Two approaches were used to identify oligonucleotide sequences that inhibit self-splicing in a transcription mixture. Prediction of potential secondary structures suggested an 8-mer complementary to one side of an internal loop in a suboptimal secondary structure (FIG. 1A). Site-directed mutations on the T. thermophila group I ribozyme have shown that formation of a similar internal loop slows folding to the active species by interfering with formation of the P3/P7 pseudoknot (Pan et al., J. Mol. BioL 280:597-609 (1998), which is hereby incorporated by reference in its entirety), as was suggested by predictions of secondary structure (Banerjee et al., Biochemistry 34:6504-6512 (1995), which is hereby incorporated by reference in its entirety). An RNA 8-mer did not bind tightly enough to stabilize the predicted misfold of the C. albicans intron. Replacing the backbone with LNA, however, provided inhibition of self-splicing with an IC50 of about 150 nM and caused misfolding of ribozyme as revealed by native gel electrophoresis and DEPC modification.

Convenient synthesis of DNA oligonucleotides (Caruthers et al., Methods Enzymol. 211:3-20 (1992); Matteucci et al., Tetrahedron Lett. 21:719-722 (1980), each of which is hereby incorporated by reference in its entirety) allowed screening to be used as a second way to identify sequences suitable for the ODMiR approach. Of 33 sequences screened at 100 μM, only 5 significantly affected self-splicing during transcription. The most effective sequence, d(TCTACGACGGCC) (SEQ ID NO: 14), also targets a region partially including the P3/P7 pseudoknot. This 12-mer also spans nucleotides involved in known tertiary interactions in group I ribozymes (Doudna et al., RNA 1:36-45 (1995); Tanner et al., RNA 3:1037-1051 (1997); Michel et al., Nature 347:578-580 (1990), each of which is hereby incorporated by reference in its entirety). Replacing the backbone with 2′-O-methyl or half the backbone with LNA provided inhibitors with IC50s of about 50 nM and 30 nM, respectively. These oligonucleotides also caused misfolding of the ribozyme as revealed by native gel electrophoresis for both (FIG. 5) and by DEPC modification for T^{L}CT^{L}AC^{L}GA^{L}CG^{L}GC^{L}C (SEQ ID NO: 3) (FIGS. 6 and 7).

The new DEPC modifications of A112 and A114 seen when ribozyme is annealed with T^{L}CT^{L}AC^{L}GA^{L}CG^{L}GC^{L}C (SEQ ID NO: 3) or ^{L}(TACCTTTC ) (SEQ ID NO: 5) are particularly interesting. By analogy to the T. thermophila group I intron, A112 forms a tertiary contact to A72, while A114 forms a U284:A114:U260 base triple (Szewczak et al., Proc. Natl. Acad. Sci. USA 96:11183-11188 (1999), which is hereby incorporated by reference in its entirety). Furthermore, when ribozyme is annealed with T^{L}CT^{L}AC^{L}GA^{L}CG^{L}GC^{L}C (SEQ ID NO: 3), A285 is modified. A285's N7 forms a tertiary contact to the 2′OH of U-3 (Pyle et al., Nature 358:123-128 (1992), which is hereby incorporated by reference in its entirety). Accessibility of A285's N7 to DEPC modification suggests that this tertiary contact is perturbed. Evidently, the docking equilibrium of the P1 helix is less favorable in the presence of d(T^{L}CT^{L}AC^{L}GA^{L}CG^{L}GC^{L}C) (SEQ ID NO: 3). Taken together, the evidence suggests that the presence of d(T^{L}CT^{L}AC^{L}GA^{L}CG^{L}GC^{L}C) (SEQ ID NO: 3) or ^{L}(TACCTTTC ) (SEQ ID NO: 5) perturbs the tertiary structure of the C. albicans group I intron.

Both approaches to identifying oligonucleotide inhibitors gave sequences expected to interfere with formation of the P3/P7 pseudoknot and with other tertiary interactions. Folding of the P3/P7 pseudoknot is known to be a slow step in formation of active ribozyme from full length transcript (Pan et al., J. Mol. Biol. 280:597-609 (1998); Banerjee et al., Biochemistry 34:6504-6512 (1995); and Zarrinkar et al., Science 265:918-924 (1994), each of which is hereby incorporated by reference in its entirety), although this does not necessarily imply that it will be slow during transcription (Pan et al., J. Mol. Biol. 280:597-609 (1998); Banerjee et al., Biochemistry 34:6504-6512 (1995); Pan et al., Proc. Natl. Acad. Sci. USA 96:9545-50 (1999), each of which is hereby incorporated by reference in its entirety). Without being bound by belief, it is believed that pseudoknots and/or other tertiary interactions may provide a general strategy for ODMiR design.

Specificity is a key issue in design of therapeutics. While 33 deoxyoligonucleotide dodecamers complementary to the C. albicans group I intron were tested, only 5 interfered with self-splicing (binding sites in P2.1, P3, P6, P7, and P9.1). Only three of these have IC50s less than 5 μM. Thus, complementarity itself is not sufficient to provide inhibition. Moreover, addition of up to 11 mM nucleotide concentration of bulk RNA from Torula Yeast does not significantly affect the IC50s of ODMiR oligomers. This suggests that oligonucleotides that inhibit an RNA by directing misfolding will not inhibit all other RNAs containing a complementary sequence. Improvements in predicting structure and in understanding of optimal targets for the ODMiR approach should eventually allow computational screening of genome sequences for designing oligonucleotides that will only affect a particular RNA. Moreover, the ODMiR approach should be applicable to any functional RNA that requires a specific secondary and/or tertiary structure. This includes RNAs that interact with proteins, mRNAs with regulatory UTRs, and catalytic RNAs such as RNase P RNA and group II introns. Thus, the ODMiR approach provides a potentially general method for targeting RNA with short oligonucleotides.

From the foregoing, it should be appreciated that the present invention provides a method for inducing non-functional misfolds into the secondary and/or tertiary structure of an RNA molecule, which but for such misfolds would primarily adopt a functional secondary and/or tertiary structure. Specifically, by way of the above example using the C albicans group I intron, ODMiR was effectively employed to cause misfolding of the intron during transcription and substantially inhibit its activity. When the intron is transcribed in the presence of ^{L}(TACCTTTC) (SEQ ID NO: 5) and T^{L}CT^{L}AC^{L}GA^{L}CG^{L}GC^{L}C (SEQ ID NO: 3), self-splicing is inhibited by 50% at about 150 and 30 nM oligonucleotide, respectively. Diethyl pyrocarbonate modification of purine N7s shows that these oligonucleotides prevent specific tertiary contacts from being formed in the P3/P7 region. ODMiRs should be applicable to many functional RNAs that require a specific secondary and/or tertiary structure.

### Example 2

### Oligonucleotide Directed Misfolding of E. coli RNase P RNA

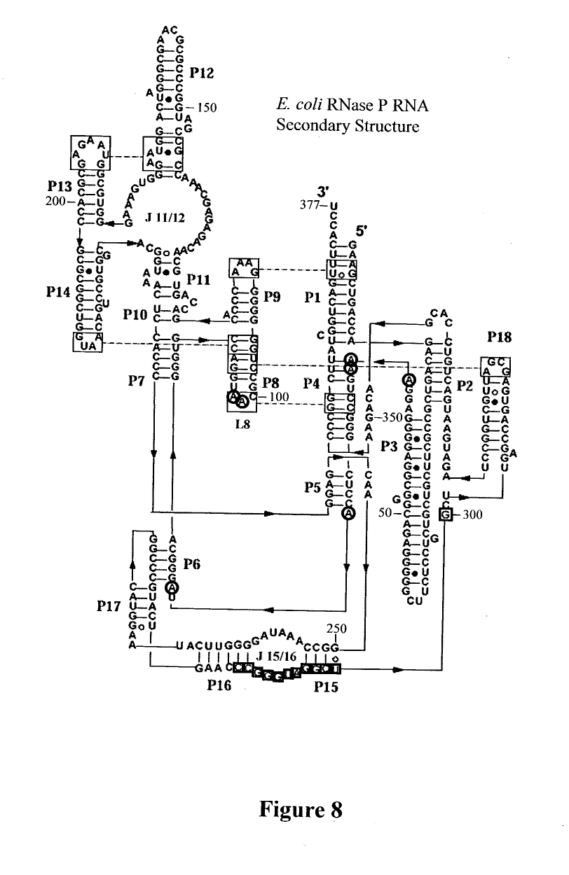

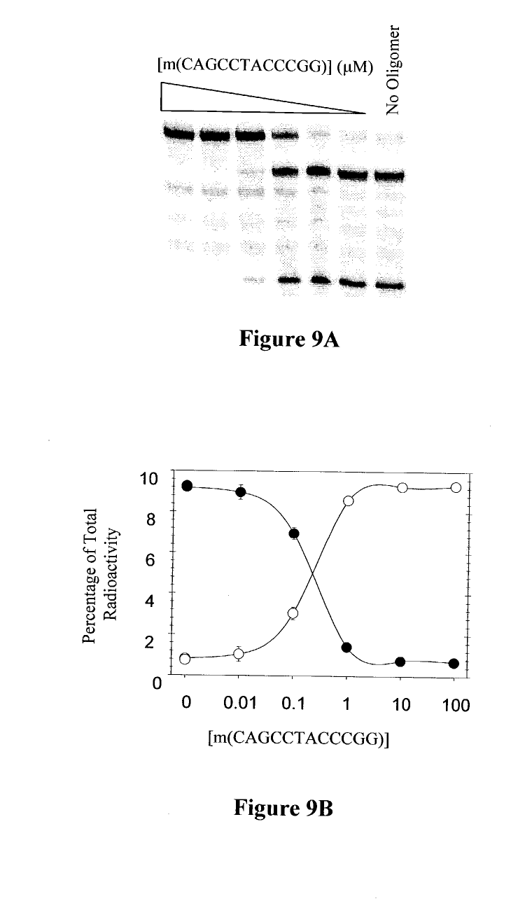

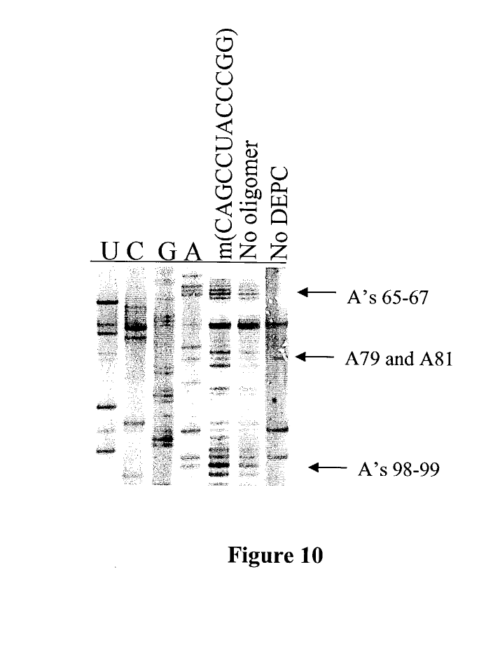

FIG. 8 (Massire et al., J. Mol. Biol. 279:773-793 (1998), which is hereby incorporated by reference in its entirety) shows the functional secondary structure of E. coli RNase P RNA. Thirty-two DNA 12-mers complementary to consecutive regions of RNase P RNA were screened for inhibition of pre-tRNA processing in a transcription mixture containing E. coli RNase P protein. Eight of these oligonucleotides inhibited at least 40% of pre-tRNA processing at 10 μM oligonucleotide concentration. Dose response curves were measured for all eight of these sequences as their 2′-O-Me analogues. The sequence m(CAGCCUACCCGG) (SEQ ID NO: 12), which is complementary to nucleotides 289-300, inhibited pre-tRNA processing most efficiently, with an IC₅₀ of about 200 nM (FIG. 9). Changing the oligonucleotide sequence to shift the binding site six residues in either direction slightly increases the IC₅₀ to ˜1 μM. Changing the sequence so that it binds nucleotides 291-302 does not significantly affect the IC₅₀, while removing 2 nucleotides from the 3′ end to give m(CAGCCUACCC) (nt 1-10 of SEQ ID NO: 12) increases the IC₅₀ to ˜1 μM. The binding site for m(CAGCCUACCCGG) (SEQ ID NO: 12) was determined experimentally by reverse transcription stops in the presence or absence of oligonucleotide. Stops are seen at the binding site, because reverse transcriptase is unable to proceed through the oligonucleotide. As expected, m(CAGCCUACCCGG) (SEQ ID NO: 12) binds to nucleotides 289-300. Differences in structure between the functional fold of RNase P RNA and the structure formed in the presence of m(CAGCCUACCCGG) (SEQ ID NO: 12) were probed by diethyl pyrocarbonate (DEPC) modification. DEPC modifies purine N7's (Doudna et al., RNA 1:36-45 (1995); Ehresmann et al., Nucleic Acids Res. 15:9109-9128 (1987), each of which is hereby incorporated by reference in its entirety), and therefore is a probe for changes in tertiary structure. As seen in FIGS. 8 and 10, m(CAGCCUACCCGG) (SEQ ID NO: 12) enhances modification at A residues 6567, 79, 81, and 98-99.

RNA function has been inhibited by small molecules (Carter et al., Nature 407:340-8 (2000); Cho et al., Biochemistry 37:4985-4992 (1998); Fourmy et al., Science 274:1367-1371 (1996); Jin et al., J. Mol. Biol 298:95-110 (2000); Kaul et al., Biochemistry 41:7695-706 (2002); Lynch et al., Structure 11:43-53 (2003); Lynch et al., J. Mol. Biol. 306:1037-1058 (2001); Lynch et al., J. Mol. Biol. 306:1023-35 (2001); Vicens et al., Chem. Biol. 9:747-55 (2002), each of which is hereby incorporated by reference in its entirety) and oligonucleotide-based therapeutics (Disney et al., Proc. Natl. Acad. Sci. USA 100:1530-1534 (2003); Galderisi et al., J. Cell. Physiol. 181:251-257(1999); Stein, Nat. Biotechnol. 19:737-738 (2001); Thayer, Chem. Eng. News 80:10 (2002), each of which is hereby incorporated by reference in its entirety). Oligonucleotide-based therapeutics can be rapidly designed and screened since much is known about molecular recognition between oligonucleotides and RNA, analogues are synthetically accessible (Freier et al., Nucleic Acids Res. 25:4429-4443 (1997), which is hereby incorporated by reference in its entirety), and their pharmacokinetic properties are likely to be similar (Crooke et al., J. Pharmacol. Exp. Ther. 277:923-937 (1996), which is hereby incorporated by reference in its entirety). In principle, insight into regions that can be targeted can be gained from secondary structure prediction (Mathews et al., RNA 5:1458-1469 (1999); Walton et al., Biotechnol. Bioeng. 65:1-9 (1999), each of which is hereby incorporated by reference in its entirety). Many RNAs function in RNA-protein complexes, however, and it is possible that the RNA-protein interaction could overcome an oligonucleotide strategy for inhibition. The results in FIG. 9 show, however, that a 12-mer oligonucleotide can trap E. coli RNase P RNA during transcription in the presence of RNase P protein such that it is unable to process a pre-tRNA.

From an oligonucleotide screen of 32 deoxyoligonucleotide 12-mers, only eight inhibited at least 40% of pre-tRNA processing at 10 μM. Only three of these sequences as 2′-O-Me analogues inhibited at least 50% of processing at 1 μM. Thus, oligonucleotide complementarity to RNase P RNA is not sufficient for inhibition.

The most efficient oligonucleotide, m(CAGCCUACCCGG) (SEQ ID NO: 12), inhibited E. coli RNase P function with an IC₅₀ of about 200 nM (FIG. 9). This oligonucleotide (approximated as an RNA oligonucleotide) is predicted by the program OligoWalk™ (Mathews et al., RNA 5:1458-1469 (1999), which is hereby incorporated by reference in its entirety) to have a total binding free energy of −16.1 kcal/mol when the RNA is allowed to re-fold. Only one other 12-mer oligonucleotide tested is predicted to bind more tightly. Of all 366 possible 12-mers, it ranks in the top 12% of oligonucleotides for favorable binding free energy. When all 12 nucleotides in RNase P RNA paired to m(CAGCCUACCCGG) (SEQ ID NO: 12) are constrained to be single stranded, the lowest free energy structure is only 7 kcal/mol less favorable than the predicted lowest free energy structure in the absence of restraints. Thus, computer algorithms may be useful for identifying sequences worth screening even when the native structure has pseudoknots, which are not allowed by the algorithm (Zuker, Science 244:48-52 (1989), which is hereby incorporated by reference in its entirety).

The m(CAGCCUACCCGG) (SEQ ID NO: 12) oligonucleotide binds to nucleotides 289-300 (3′ side of Jl 5/16) as designed. It base pairs with a number of residues that have been implicated in pre-tRNA recognition, including G291, G292, and U294. In the current model, G291 forms a base triple with G259 in RNase P RNA and A76 in pre-tRNA; G292 forms a base triple with A258 in RNase P RNA and C75 in pre-tRNA; and U294 in RNase P RNA binds to R73 in pre-tRNA, where R is A or G (Heide et al., RNA 5:102-116 (1999); Heide et al., RNA 7:958-968 (2001); and Kirsebom et al., EMBO J. 13:4870-4876 (1994), each of which is hereby incorporated by reference in its entirety). There is evidence that J15/16 is also a metal ion binding site (Ciesiolka et al., Eur. J. Biochem. 219:49-56 (1994); Kazakov et al., Proc. Natl. Acad. Sci. USA 88:9193-9197 (1991); Kufel et al., J. Mol. Biol. 244:511-521 (1994); Zito et al., Nucleic Acids Res. 21:5916-5920 (1993), each of which is hereby incorporated by reference in its entirety). Part of the mode of inhibition may be competing with the pre-tRNA for this binding site or interfering with metal ion binding. Structure probing with DEPC, however, shows a significant structural rearrangement in L8 (A's 98-99), J5/6 (A79 and A81), and the single stranded region between P3 and P4 (A's 65-67) (FIGS. 8 and 10).

A's 65-67 are part of the pseudoknot containing P4 (Haas et al., Science 254:853-856 (1991), which is hereby incorporated by reference in its entirety), and are important for catalytic activity (Kazantsev et al., RNA 4:937-947 (1998), which is hereby incorporated by reference in its entirety). A's 98 and 99 in L8 are predicted to be involved in a tertiary interaction with P4 (Massire et al., J. Mol. Biol. 279:773-793 (1998), which is hereby incorporated by reference in its entirety). Thus, increased modification of A's 65-67, 98, and 99 by DEPC suggests a structural rearrangement of part of the P4 pseudoknot. A's 79 and 81 are in close proximity to the four base pairs of P6, which is also part of a pseudoknot (James et al., Cell 52:19-26 (1988), which is hereby incorporated by reference in its entirety). Deletion studies of RNase P RNA show that disruption of P6 increases the K_{M} for pre-tRNA (Darr et al., Biochemistry 31:328-333 (1992), which is hereby incorporated by reference in its entirety).

Improvements to secondary structure prediction and in understanding how large RNAs fold should eventually allow for selection of ODMiR targets from entire genomes. Such RNAs may include RNAs with secondary and/or tertiary structures important for catalysis, regulation of translation, localization, or interaction with other biomolecules.

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A method of modifying the function of an RNA molecule comprising:
providing an oligonucleotide; and
binding the oligonucleotide to an RNA molecule that possesses an activity at least partially dependent on secondary and/or tertiary folding thereof into an active conformation, wherein said binding occurs at a site of the RNA molecule that prevents folding thereof into the active conformation and thereby modifies the function of the RNA molecule.

2. The method according to 1 wherein the RNA molecule is selected from the group consisting of rRNAs; mRNAs that possess an untranslated region that regulates translation, localization, or RNA stability; RNase P; splicing introns; ribozymes; and mRNAs possessing internal ribosome entry sites (IRES).

3. The method according to claim 2 wherein the RNA molecule is an rRNA.

4. The method according to claim 2 wherein the RNA molecule is an mRNA that possesses an untranslated region that regulates translation, localization, or RNA stability.

5. The method according to claim 2 wherein the RNA molecule is an RNase P.

6. The method according to claim 2 wherein the RNA molecule is a splicing intron selected from the group of Group I introns and Group II introns.

7. The method according to claim 2 wherein the RNA molecule is a ribozyme.

8. The method according to claim 2 wherein the RNA molecule is an mRNA possessing an IRES.

9. The method according to claim 1 wherein said binding causes the RNA molecule to adopt a conformation characterized by reduced activity.

10. The method according to claim 9 wherein the conformation characterized by reduced activity is predicted to have a free energy state that is not more than the greater of either about 10 percent or about 10 kcal/mole higher than the lowest free energy state of the RNA molecule as predicted using an RNA folding software program.

11. The method according to claim 1 wherein said binding occurs during transcription of the RNA molecule.

12. The method according to claim 1 wherein said binding occurs prior to completion of secondary and/or tertiary folding of the RNA molecule into the active conformation.

13. The method according to claim 1 wherein said binding occurs in vitro.

14. The method according to claim 1 wherein said binding occurs in vivo.

15. The method according to claim 1 wherein the oligonucleotide is less than 100 nucleotides in length.

16. The method according to claim 1 wherein the oligonucleotide comprises one or more modified bases, one or more modified sugars, one or more modified backbones, or combinations thereof that enhance the affinity of the oligonucleotide to the site of the RNA molecule.

17. A method of stabilizing an RNA molecule in a substantially inactive conformation comprising:
binding an oligonucleotide to an RNA molecule at a site of the RNA molecule that causes the RNA molecule to adopt a substantially inactive conformation that is distinct of an active conformation thereof,
wherein the RNA molecule is initially stabilized in the substantially inactive conformation by said binding.

18. The method according to 17 wherein the RNA molecule is selected from the group consisting of rRNAs; mRNAs that possess an untranslated region that regulates translation, localization, or RNA stability; RNase P; splicing introns; ribozymes; and mRNAs possessing internal ribosome entry sites (IRES).

19. The method according to claim 18 wherein the RNA molecule is an rRNA.

20. The method according to claim 18 wherein the RNA molecule is an mRNA that possesses an untranslated region that regulates translation, localization, or RNA stability.

21. The method according to claim 18 wherein the RNA molecule is an RNase P.

22. The method according to claim 18 wherein the RNA molecule is a splicing intron selected from the group of Group I introns and Group II introns.

23. The method according to claim 18 wherein the RNA molecule is a ribozyme.

24. The method according to claim 18 wherein the RNA molecule is an mRNA possessing an IRES.

25. The method according to claim 17 wherein the substantially inactive conformation is predicted to have a free energy state that is not more than the greater of either about 10 percent or about 10 kcal/mole higher than the lowest free energy state of the RNA molecule as predicted using an RNA folding software program.

26. The method according to claim 17 wherein said binding occurs during transcription of the RNA molecule.

27. The method according to claim 17 wherein said binding occurs prior to completion of secondary and/or tertiary folding of the RNA molecule into the active conformation.

28. The method according to claim 17 wherein said binding occurs in vitro.

29. The method according to claim 17 wherein said binding occurs in vivo.

30. The method according to claim 17 wherein the oligonucleotide is less than 100 nucleotides in length.

31. The method according to claim 17 wherein the oligonucleotide comprises one or more modified bases, one or more modified sugars, one or more modified backbones, or combinations thereof that enhance the affinity of the oligonucleotide to the site of the RNA molecule.

32. An isolated oligonucleotide comprising a nucleotide sequence that binds to at least one domain of an RNA molecule that requires folding to achieve an active conformation that includes a secondary and/or tertiary structure, wherein binding of the oligonucleotide to the RNA molecule inhibits formation of the secondary and/or tertiary structure.

33. The isolated oligonucleotide according to claim 32 the RNA molecule is selected from the group consisting of rRNAs; mRNAs that possess an untranslated region that regulates translation, localization, or RNA stability; RNase P; splicing introns; ribozymes; and mRNAs possessing internal ribosome entry sites (IRES).

34. The isolated oligonucleotide according to claim 33 wherein the RNA molecule is a Group I splicing intron.

35. The isolated oligonucleotide according to claim 34 wherein the Group I splicing intron is a Candida Group I splicing intron.

36. The isolated oligonucleotide according to claim 35 wherein the at least one domain is a P2.1 domain, P3 domain, P6 domain, P7 domain, or P9.1 domain.

37. The isolated oligonucleotide according to claim 36 wherein the oligonucleotide comprises the nueleotide sequence of
TACCTTTC, wherein one or more of the bases are modified by propynylation, alkylation, or by the presence of an LNA base; or
TCTACGACGGCC, wherein one or more of the bases are modified by propynylation, alkylation, or by the presence of an LNA base.

38. The isolated oligonucleotide according to claim 37 wherein the oligonucleotide is selected from the group of ^{L}(TACCTTTC), T^{L}CT^{L}AC^{L}GA^{L}CG^{L}GC^{L}C, and an 2′-O-methyl derivative of TCTACGACGGCC.

39. The isolated oligonucleotide according to claim 33 wherein the RNA molecule is an RNase P.

40. The isolated oligonucleotide according to claim 39 wherein the RNase P is an E. coli RNase P.

41. The isolated oligonucleotide according to claim 40 wherein the at least one domain is an L8 domain, J15/16 domain, or P4 domain.

42. The isolated oligonucleotide according to claim 41 wherein the oligonucleotide has the nucleic acid sequence of CAGCCUACCCGG.

43. The isolated oligonucleotide according to claim 32 wherein the oligonucleotide is less than 100 nucleotides in length.

44. The isolated oligonucleotide according to claim 32 wherein the oligonucleotide is less than 50 nucleotides in length.

45. The isolated oligonucleotide according to claim 32 wherein the oligonucleotide comprises one or more modified bases, one or more modified sugars, one or more modified backbones, or combinations thereof.

46. The isolated oligonucleotide according to claim 32 wherein the oligonucleotide is formed of RNA.

47. The isolated oligonucleotide according to claim 32 wherein the oligonucleotide is formed of DNA.

48. A method of disrupting survivability of a pathogen, the method comprising:
providing an oligonucleotide that binds to an RNA molecule of the pathogen, which RNA molecule is characterized by secondary and/or tertiary folding to achieve an active conformation required for pathogen survivability; and
binding the oligonucleotide to the RNA molecule, whereby said binding causes the RNA molecule to misfold, thereby disrupting the activity of the RNA molecule and survivability of the pathogen.

49. The method according to claim 48 wherein the RNA molecule is selected from the group consisting of rRNAs; mRNAs that possess an untranslated region that regulates translation, localization, or RNA stability; RNase P; splicing introns; ribozymes; and mRNAs possessing internal ribosome entry sites (IRES).

50. The method according to claim 48 wherein said binding occurs during transcription of the RNA molecule.

51. The method according to claim 48 wherein said binding occurs prior to completion of secondary and/or tertiary folding of the RNA molecule into the active conformation.

52. The method according to claim 48 wherein the pathogen is in vivo.

53. The method according to claim 48 wherein the pathogen is ex vivo.

54. The method according to claim 48 wherein the oligonucleotide is less than 100 nucleotides in length.

55. The method according to claim 48 wherein the oligonucleotide comprises one or more modified bases, one or more modified sugars, one or more modified backbones, or combinations thereof.

56. A method of treating or preventing pathogen infection in a patient, the method comprising:
providing an oligonucleotide that binds to an RNA molecule of a pathogen, which RNA molecule is characterized by secondary and/or tertiary folding to achieve an active conformation required for pathogen survivability; and
administering the oligonucleotide to a patient under conditions effective to cause uptake of the oligonucleotide by the pathogen or patient cells infected with the pathogen, whereby the oligonucleotide binds to the RNA molecule and said binding causes the RNA molecule to misfold, thereby disrupting survivability of the pathogen to treat or prevent pathogen infection in the patient.

57. The method according to claim 56 wherein the RNA molecule is selected from the group consisting of rRNAs; mRNAs that possess an untranslated region that regulates translation, localization, or RNA stability; RNase P; splicing introns; ribozymes; and mRNAs possessing internal ribosome entry sites (IRES).

58. The method according to claim 56 wherein said binding occurs during transcription of the RNA molecule.

59. The method according to claim 56 wherein said binding occurs prior to completion of secondary and/or tertiary folding of the RNA molecule into the active conformation.

60. The method according to claim 56 wherein the oligonucleotide is less than 100 nucleotides in length.

61. The method according to claim 56 wherein the oligonucleotide comprises one or more modified bases, one or more modified sugars, one or more modified backbones, or combinations thereof.

62. The method according to claim 56 wherein the oligonucleotide is administered in a composition comprising a pharmaceutically acceptable carrier.

63. The method according to claim 56 wherein said administering is carried out orally, parenterally, subcutaneously, intravenously, intramuscularly, intraperitoneally, by intranasal instillation, by inhalation, by implantation, by intracavitary or intravesical instillation, intraocularly, intraarterially, intralesionally, transdermally, by application to mucous membranes, rectally, vaginally, or topically.

64. A method of disrupting survival or proliferation of a cancer cell, the method comprising:
providing an oligonucleotide that binds to an mRNA molecule overexpressed in a cancer cell, wherein overexpression of the mRNA is required for survival or proliferation of the cancer cell; and
binding the oligonucleotide to the mRNA molecule, whereby said binding causes the mRNA molecule to misfold, thereby disrupting translation of the mRNA molecule and either survival or proliferation of the cancer cell.

65. The method according to claim 64 wherein said binding occurs during transcription of the mRNA molecule.

66. The method according to claim 64 wherein the cancer cell is in vivo.

67. The method according to claim 64 wherein the cancer cell is ex vivo.

68. The method according to claim 64 wherein the oligonucleotide is less than 100 nucleotides in length.

69. The method according to claim 64 wherein the oligonucleotide comprises one or more modified bases, one or more modified sugars, one or more modified backbones, or combinations thereof.

70. A method of treating a cancerous condition in a patient, the method comprising:
providing an oligonucleotide that binds to an mRNA molecule overexpressed in a cancer cell, wherein overexpression of the mRNA is required for survival or proliferation of the cancer cell; and
administering the oligonucleotide to a patient under conditions effective to cause uptake of the oligonucleotide by the cancer cell, whereby the oligonucleotide binds to the mRNA molecule and said binding causes the mRNA molecule to misfold, thereby disrupting translation of the mRNA molecule and either survival or proliferation of the cancer cell, which treats the cancerous condition.

71. The method according to claim 70 wherein said binding occurs during transcription of the mRNA molecule.

72. The method according to claim 70 wherein the oligonucleotide is less than 100 nucleotides in length.

73. The method according to claim 70 wherein the oligonucleotide comprises one or more modified bases, one or more modified sugars, one or more modified backbones, or combinations thereof.

74. The method according to claim 70 wherein the oligonucleotide is administered in a composition comprising a pharmaceutically acceptable carrier.

75. The method according to claim 70 wherein said administering is carried out orally, parenterally, subcutaneously, intravenously, intramuscularly, intraperitoneally, by intranasal instillation, by inhalation, by implantation, by intracavitary or intravesical instillation, intraocularly, intraarterially, intralesionally, transdermally, by application to mucous membranes, rectally, vaginally, or topically.

76. A method of making an oligonucleotide that directs misfolding of an RNA molecule into a conformation having modified activity, said method comprising:
predicting the folding structure of an RNA molecule that is characterized by secondary and/or tertiary folding to achieve an active conformation;
designing an oligonucleotide to hybridize to the RNA molecule at a site critical for the secondary and/or tertiary folding; and
determining whether binding of the oligonucleotide at the site modifies folding of RNA molecule and thereby modifies the activity of the RNA molecule.

77. The method according to claim 76 wherein said designing comprises introducing one or more modified sugars, one or more modified backbones, or combinations thereof in the nucleotide sequence to increase the affinity of the oligonucleotide for the site of the RNA molecule.

78. The method according to claim 76 wherein said determining is carried out using an RNA folding software program.

79. The method according to claim 76 wherein said determining is carried out by identifying altered mobility of the RNA molecule during native gel electrophoresis.

80. The method according to claim 76 wherein said determining is carried out by exposing the RNA molecule to a modification agent in the presence or absence of the oligonucleotide and detecting nucleotides of the RNA molecule that are modified by the modification agent in the presence or absence of the oligonucleotide, wherein differences in modified nucleotides indicates that binding of the oligonucleotide to the RNA molecule modifies secondary and/or tertiary structure of the RNA molecule.

81. A DNA construct comprising a DNA molecule that encodes the oligonucleotide of claim 46.

82. An expression vector comprising a DNA molecule that encodes the oligonucleotide of claim 46.

83. A host cell comprising the expression vector according to claim 82.

84. The method according to claim 48 wherein the pathogen is a virus, bacteria, yeast, or fungus.

85. The method according to claim 56 wherein the pathogen is a virus, bacteria, yeast, or fungus.
